(19) United States
(12) Patent Application Publication
(10) Pub. No.: US 2016/0115473 A1
(43) Pub. Date: Apr. 28, 2016

(54) MULTIFUNCTIONAL OLIGONUCLEOTIDES

(71) Applicant: ABBOTT MOLECULAR INC., DES PLAINES, IL (US)

(72) Inventor: Dae Hyun Kim, Des Plaines, IL (US)

(21) Appl. No.: 14/826,951

(22) Filed: Aug. 14, 2015

Related U.S. Application Data
(60) Provisional application No. 62/037,331, filed on Aug. 14, 2014.

(51) Int. Cl. C12N 15/10

(52) U.S. Cl. CPC C12N 15/1068

(57) ABSTRACT

Provided herein is technology relating to the manipulation and characterization of nucleic acids and particularly, but not exclusively, to methods and compositions relating to oligonucleotide primers and probes for amplifying, quantifying, and sequencing nucleic acids.

### CROSS-REFERENCE TO RELATED APPLICATIONS

The present application claims priority to U.S. Provisional Application Ser. No. 62/037,331 filed Aug. 14, 2014, the entirety of which is incorporated by reference herein.

### FIELD

Provided herein is technology relating to the manipulation and characterization of nucleic acids and particularly, but not exclusively, to methods and compositions relating to oligonucleotide primers and probes for amplifying, quantifying, and sequencing nucleic acids.

### BACKGROUND

Molecular diagnostics using DNA sequencing is an important element of medical research and clinical practice. The incorporation of DNA sequencing into medical care has largely been driven by the development of next-generation sequencing (NGS) technologies, which provide a low-cost and high-throughput means for determining nucleic acid sequences. For example, sequence data has found use in diagnostics for cancer, infectious diseases, companion drugs, and hereditary conditions. It has become evident that NGS has broad application in medicine and the emergent provision of personalized medical care will increase the demand for sequencing at all scales (e.g., from SNPs to genes, chromosomes, and entire genomes).

Most NGS platforms require a sequencing library as input. While each particular NGS platform has its own specific requirements for the sequencing library, workflows for producing sequencing libraries from nucleic acid samples typically include steps for quantifying the nucleic acid sample and adding platform-specific adaptors to the ends of the nucleic acids in the sample. The adaptors are a prerequisite for introduction of the library into the NGS workflow. In particular, the adaptors provide sites to initiate sequencing of the individual nucleic acids with common platform-specific primers. Accurate quantification of the sequencing library is critical for providing a concentration normalized library into the NGS workflow to produce high quality sequence data.

In particular, one existing method first generates the amplicon using traditional PCR and typical linear primers, followed by enzymatically ligating an adaptor comprising the platform-dependent (e.g., “universal”) sequence to the amplicons. Some other existing technologies involve the use of “fusion primers”, which have an amplicon-specific priming sequence flanked by the platform-dependent (e.g., “universal”) sequence on the 5′ side.

These current NGS work-flows involve multiple steps and/or reactions to prepare sequencing libraries. For example, extant amplification-based workflows incorporate separate amplification, quantification, and adaptor ligation steps, with purification, quality control, and quantification steps often occurring between each of these steps. Performing these multiple DNA fragment processing, purification, and quality control procedures requires extensive hands-on time, prolonged work-flow time, increased use of reagents, and more opportunities for user error. Consequently, these factors contribute to limit sample preparation throughput and increase the cost per sample preparation in terms of both reagent cost and lab personnel time and effort. Ultimately, the per-base cost of a DNA sequence read is increased. In addition, the overall data output is sub-optimal because sequence output is limited not by the sequencing capacity of the instrumentation, but by the provision of samples for analysis.

In addition, existing technologies comprising use of “fusion” primers introduce off-target amplicons into the amplicon pool, thus affecting the efficiency and reliability of library generation. In particular, the platform-dependent (e.g., “universal” sequences) are exposed during all stages of sample work-up, preparation, and thermal cycling. Thus, when amplification is performed using the fusion primers, amplicons are generated comprising universal sequences incorporated at the ends of the amplicons and subsequent complex hybridization interactions (e.g., amplicon-amplicon and amplicon-fusion primer) produce unwanted amplification products. One result is the production of non-target sequences. Another problem is that these inefficiencies limit the scalability and use of the existing technology in multiplexed protocols for library generation and sequencing.

### SUMMARY

Accordingly, provided herein is technology related, in some embodiments, to manipulating nucleic acids. In some embodiments, the technology relates to producing NGS sequencing libraries. The technology provides an efficient “one-step/one-tube” generation and quantification of an amplicon library for NGS. Hands-on time is less than existing technologies, e.g., because the technology is associated with fewer steps to perform. For example, in some embodiments the hands-on time associated with the present technology is limited to preparing a single PCR reaction, which can be completed in approximately 15 minutes. Further, the general total overall work-flow is associated with assembling and thermal cycling a single amplification reaction and a subsequent product purification step, which together take approximately 2 hours or less. The technology provides multiplexing capabilities that are associated with additional reductions in reagent costs and increases in sample preparation throughput. Also, due to a significantly more simplified work-flow than existing technologies, the entire work-flow is amenable for automation. Some embodiments are feasible with less complex and less expensive automation systems than extant technologies.

Existing work-flows for NGS amplicon-based library generation are complex, expensive, and time-intensive, and thus have limited applicability in clinical and/or diagnostic lab settings. In contrast, the technology provided herein finds use in clinical and/or diagnostic lab settings because it is a technology that is easy to perform, has a low cost, and produces results with a fast turnaround. The technology provides for the robust production of multi-amplicon libraries in a single tube. The libraries are ready for input into a NGS system work-flow with minimal hands-on time and with significant decrease in overall work-flow time and cost. The technology is easily automatable, which provides additional increases in efficiencies.

In particular, the technology relates to the design and use of oligonucleotides that form a “hairpin” or “step-loop” structure. In some embodiments, the technology provides oligonucleotides comprising a portion that forms a double-stranded element through intra-molecular interactions and a portion that remains in a single stranded form, e.g., for hybridization to a complementary (e.g., target) sequence, e.g., to serve as a primer for amplification. In particular embodiments, the oligonucleotides comprise a first self-complementary region and a second self-complementary region that hybridize to each other (e.g., through intramolecular interaction) to form the double-stranded element.

In some embodiments, the oligonucleotides comprise a single-stranded loop region (e.g., between the first self-complementary region and the second self-complementary region), one or more fluorescent moieties (e.g., a fluorescent moiety and/or a quenching moiety), and/or a moiety that is resistant to degradation (e.g., by an enzyme such as an exonuclease, e.g., a 5′ to 3′ exonuclease, or an enzyme (e.g., a polymerase) comprising exonuclease, e.g., a 5′ to 3′ exonuclease, activity). In some embodiments, the single-stranded loop region comprises a PEG (polyethylene glycol) linker. Further, in some embodiments, a PEG linker connects the first self-complementary region and the second self-complementary region.

In some embodiments, the oligonucleotides comprise a fluorescent moiety and a quencher moiety. The fluorescent moiety and the quencher moiety can by located in various places, without limitation, on the oligonucleotides. For example, embodiments provide that the first self-complementary region comprises a fluorescent moiety and the second self-complementary region comprises a quenching moiety. Embodiments provide that the second self-complementary region comprises a fluorescent moiety and the first self-complementary region comprises a quenching moiety.

In some preferred embodiments, a fluorescent moiety and a quenching moiety are present on the same self-complementary region of the double-stranded element (e.g., the fluorescent moiety and the quenching moiety are both on the same strand of the hairpin duplex, e.g., the first self-complementary region comprises a fluorescent moiety and a quenching moiety or the second self-complementary region comprises a fluorescent moiety and a quenching moiety).

In some embodiments, the oligonucleotides according to the technology comprise a fluorescent moiety and a quencher moiety that are appropriately placed in space so that the quencher moiety quenches the fluorescence of the fluorescent moiety (e.g., when the fluorescent moiety is excited, e.g., by exposing the fluorescent moiety to electromagnetic radiation of an appropriate (e.g., excitation) wavelength). In some embodiments, degradation of the first self-complementary region or degradation of the second self-complementary region separates the quencher moiety from the fluorescent moiety so that the quencher moiety does not quench the fluorescence of the fluorescent moiety (e.g., when the fluorescent moiety is excited, e.g., by exposing the fluorescent moiety to electromagnetic radiation of an appropriate (e.g., excitation) wavelength). For example, some embodiments comprise use of a polymerase (e.g., a Taq polymerase) and oligonucleotide primers provided herein for a PCR. As the polymerase (e.g., Taq polymerase) synthesizes a nascent strand and encounters the 5′ end of the double-stranded region, the 5′ to 3′ exonuclease activity of the polymerase degrades the first self-complementary region or the second self-complementary region. Degradation of the first self-complementary region or the second self-complementary region releases the fluorophore and/or quencher from it and breaks the close proximity of the fluorescent moiety to the quencher, thus relieving the quenching effect and promoting the fluorescent moiety to fluoresce. In some embodiments, the fluorescence detected in a quantitative PCR thermal cycler is directly proportional to the fluorescent moiety released and the amount of target DNA (e.g., amplicon and/or template) present in the PCR.

In some embodiments, the oligonucleotides comprise a blocker (e.g., nuclease-resistant) moiety that is resistant to degradation, e.g., by an enzyme (e.g., an enzyme having exonuclease activity (e.g., an exonuclease enzyme or a polymerase enzyme comprising an exonuclease activity)). In some embodiments, the single-stranded loop region comprises a blocker moiety. In some embodiments, the first self-complementary region or the second self-complementary region comprises the blocker moiety. In some embodiments, the blocker moiety defines a junction between the single-stranded loop region and the first self-complementary region or between the single-stranded loop region and the second self-complementary region. In some embodiments, the blocker moiety is a phosphorothioate bond or a nucleotide analog. In some embodiments, the blocker moiety blocks the progress of an enzyme (e.g., a polymerase) having 5′ to 3′ exonuclease activity. In some embodiments, blocking the progress of an enzyme (e.g., a polymerase) having 5′ to 3′ exonuclease activity defines a known end sequence or provides a defined end sequence of a nucleic acid such as an amplicon produced according to the technology, e.g., an amplicon comprising a user-defined adaptor (e.g., an adaptor comprising, e.g., a tag (e.g., comprising a linker, index, capture sequence, restriction site, primer binding site, antigen, and/or other functional site) and/or a universal sequence (e.g., a platform-specific sequence)). In some embodiments associated with use of a proofreading polymerase (e.g., a high-fidelity polymerase) comprising a 3′ exonuclease activity but lacking a 5′ exonuclease activity, the oligonucleotides comprise a PEG linker and the PEG-DNA junction stops polymerase extension.

In some embodiments, the oligonucleotides find use in the amplification of nucleic acids. For example, in some embodiments the oligonucleotides find use in a polymerase chain reaction (PCR) to produce an amplification product. In some embodiments, the oligonucleotides find use to produce an amplification product (e.g., an amplicon) comprising two portions:
1) a first portion comprising, derived from, and/or complementary to the target template; and
2) a second portion comprising a user-defined adaptor (e.g., an adaptor comprising a tag (e.g., a tag comprising a linker, index, capture sequence, restriction site, primer binding site, antigen, and/or other functional site) and/or comprising a universal sequence (e.g., comprising a platform-dependent sequence)).

That is, embodiments of the technology produce amplicons comprising a target sequence concatenated to a user-defined functional sequence such as an adaptor as described herein.

Furthermore, the technology provides real-time relative quantification of the amplification products. In some embodiments, real-time relative quantification of the amplification products occurs without a separate labeled probe, e.g., as is used in a real-time quantitative PCR comprising a hydrolysis probe (e.g., a Taqman probe). Accordingly, the technology (e.g., oligonucleotides and methods using them) provides a quantified “one-step” generation of amplicons comprising target sequence and a user-defined adaptor when used as primers in a PCR. This technology simplifies the work-flow of NGS sequencing library generation.

Accordingly, provided herein are embodiments of a hairpin oligonucleotide. In some embodiments the hairpin oligonucleotide comprises a first portion comprising, derived from, and/or complementary to the target template (e.g., an amplicon-specific priming segment); and a second portion comprising a user-defined adaptor.

In some embodiments the hairpin oligonucleotide comprises a first portion comprising, derived from, and/or complementary to the target template (e.g., an amplicon-specific priming segment); and a second portion comprising a user-defined adaptor comprising a tag.

In some embodiments the hairpin oligonucleotide comprises a first portion comprising, derived from, and/or complementary to the target template (e.g., an amplicon-specific priming segment); and a second portion comprising a user-defined adaptor comprising a universal sequence (e.g., comprising a platform-dependent sequence)).

In some embodiments the hairpin oligonucleotide comprises a first portion comprising, derived from, and/or complementary to the target template (e.g., an amplicon-specific priming segment); and a second portion comprising a user-defined adaptor comprising a tag (e.g., a tag comprising a linker, index, capture sequence, restriction site, primer binding site, antigen, and/or other functional site) and a universal sequence (e.g., comprising a platform-dependent sequence)).

In some embodiments, the hairpin oligonucleotide comprises a single-stranded region comprising an amplicon-specific priming segment and a double-stranded duplex region comprising a first self-complementary region hybridized to a second self-complementary region.

In some embodiments, the hairpin oligonucleotide comprises a single-stranded region comprising an amplicon-specific priming segment; a double-stranded duplex region comprising a first self-complementary region hybridized to a second self-complementary region; and a single-stranded loop region.

In some embodiments, the hairpin oligonucleotide comprises a single-stranded region comprising an amplicon-specific priming segment; a double-stranded duplex region comprising a first self-complementary region hybridized to a second self-complementary region; and a PEG linker.

In some embodiments, the hairpin oligonucleotide comprises a single-stranded region comprising an amplicon-specific priming segment; a double-stranded duplex region comprising a first self-complementary region hybridized to a second self-complementary region; a single-stranded loop region; a blocker moiety; a fluorescent moiety; and a quenching moiety, wherein the second self-complementary region comprises the fluorescent moiety and the quenching moiety.

In some embodiments, the hairpin oligonucleotide comprises a single-stranded region comprising an amplicon-specific priming segment; a double-stranded duplex region comprising a first self-complementary region hybridized to a second self-complementary region; a single-stranded loop region; and a blocker moiety.

The hairpin oligonucleotides described herein comprise, in various embodiments, segments, elements, features, and/or sequences that provide desirable characteristics to the hairpin oligonucleotides. For example, in some embodiments the hairpin oligonucleotides comprise an adaptor. In some embodiments, the adaptor in turn comprises a tag; in some embodiments, the tag comprises a linker, index, capture sequence, restriction site, primer binding site, antigen, and/or or other functional site. In some embodiments, the adaptor comprises a universal sequence (e.g., a platform-dependent sequence).

The technology is not limited in the placement of the tag. In some particular embodiments, the tag is positioned between the amplicon-specific priming segment and the double-stranded region (see, e.g., FIG. 1). However, the tag can be positioned in various locations within the primary structure of the hairpin oligonucleotide. In some embodiments, the tag sequence is within and/or overlaps one or more other segments, elements, features, and/or sequences of the hairpin oligonucleotide. For example, in some embodiments the single-stranded loop region comprises a tag.

Embodiments of the hairpin oligonucleotides comprise a blocker moiety that is resistant to nuclease activity. For example, in some embodiments the blocker moiety is exonuclease resistant, e.g., resistant to 5′ to 3′ exonuclease activity. The technology is not limited in the type, structure, or composition of the blocker moiety provided that the blocker moiety is nuclease resistant. An exemplary blocker moiety provides a nuclease resistant bond between adjacent nucleotides in a nucleic acid, e.g., in some embodiments the blocker moiety is a phosphorothioate bond. In some embodiments, the blocker moiety is a peptide-nucleic acid linkage. In some embodiments the blocker moiety is at or near the junction of the single-stranded loop region and the double-stranded duplex region.

The technology is not limited in the type, structure, or composition of the fluorescent moiety. Non-limiting examples of fluorescent moieties include dyes that can be synthesized or obtained commercially (e.g., Operon Biotechnologies, Huntsville, Ala.). A large number of dyes (greater than 50) are available for application in fluorescence excitation applications. These dyes include those from the fluorescein, rhodamine, AlexaFluor, Bodipy, Coumarin, and Cyanine dye families. Specific examples of fluorophores include, but are not limited to, FAM, TET, HEX, Cy3, TMR, ROX, VIC (e.g., from Life Technologies), Texas red, LC red 640, Cy5, and LC red 705. In some embodiments, dyes with emission maxima from 410 nm (e.g., Cascade Blue) to 775 nm (e.g., Alexa Fluor 750) are available and can be used. Of course, one of ordinary skill in the art will recognize that dyes having emission maxima outside these ranges may be used as well. In some cases, dyes ranging between 500 nm to 700 nm have the advantage of being in the visible spectrum and can be detected using existing photomultiplier tubes. In some embodiments, the broad range of available dyes allows selection of dye sets that have emission wavelengths that are spread across the detection range. Detection systems capable of distinguishing many dyes are known in the art.

Further, the technology is not limited in the type, structure, or composition of the quenching moiety. Exemplary quenching moieties include a Black Hole Quencher, an Iowa Black Quencher, and derivatives, modifications thereof, and related moieties. Exemplary quenching moieties include BHQ-0, BHQ-1, BHQ-2, and BHQ-3.

The double-stranded region of the hairpin oligonucleotide may comprise hybridized segments that are completely complementary or that are not completely complementary provided that the duplex forms at a desirable temperature and reaction conditions as described herein. As such, some particular embodiments provide that the double-stranded duplex region comprises at least one mismatch (e.g., a mismatch, e.g., 1, 2, 3, 4, 5, 6, 7, 8, 9, 10 or more mismatches).

The hairpin oligonucleotides may assume different conformations. For example, in some embodiments the first self-complementary region and the second self-complementary region are not hybridized at or above a denaturing temperature (e.g., above 89, 90, 91, 92, 93, 94, 95, 96, or 97° C.) in an amplification reaction. In some embodiments, the first self-complementary region and the second self-complementary region are hybridized below the denaturing temperature (e.g., at approximately 65 to 80° C., e.g., 65, 66, 67, 68, 69, 70, 71, 72, 73, 74, 75, 76, 77, 78, 79, or 80° C.) in an amplification reaction. See, e.g., FIG. 2.

Embodiments of the technology relate to reaction mixtures comprising hairpin oligonucleotides as described herein. For example, some embodiments provide a reaction mixture comprising a hairpin oligonucleotide as described herein and a template, wherein the single-stranded region (e.g., the primer region) is hybridized to the template and the first self-complementary region is hybridized to the second self-complementary region.

Also contemplated are amplicons produced from the hairpin oligonucleotides provided herein. Particular embodiments provide an amplicon comprising a first portion comprising, derived from, and/or complementary to the target template and a second portion comprising a user-defined adaptor.

Some embodiments are related to amplicons comprising a tag (e.g., comprising a linker, index, capture sequence, restriction site, primer binding site, antigen, and/or other functional site) and/or a universal sequence (e.g., platform-dependent sequence). In some embodiments an amplicon comprises a tag after a portion of the hairpin oligonucleotide-derived portion of the amplicon has been hydrolyzed by a nuclease activity (e.g., an exonuclease activity of a polymerase). For example, some embodiments provide an amplicon comprising a sequence comprising, derived from, and/or complementary to the target template; a tag; and the first self-complementary sequence derived from a hairpin oligonucleotide as described herein, but wherein the amplicon lacks: the second self-complementary sequence derived from the hairpin oligonucleotide; the fluorescent moiety; and the quencher moiety (see, e.g., the amplicon in FIG. 3 after Step 4). Such amplicons do not comprise the fluorescent moiety due to the nuclease activity that releases the fluorescent moiety into solution. As such, embodiments provide a reaction mixture comprising an amplicon as described above (e.g., an amplicon comprising a sequence comprising, derived from, and/or complementary to the target template; a tag; and the first self-complementary sequence derived from a hairpin oligonucleotide as described herein) and a free fluorescent moiety. In some embodiments, such reaction mixtures further comprise a polymerase comprising an exonuclease activity (e.g., a 5′ to 3′ exonuclease activity) or a polymerase (e.g., a high-fidelity polymerase) comprising a proof-reading activity, a 3′ exonuclease activity, and/or a strand displacement activity, but lacking a 5′ exonuclease activity. Related embodiments further comprise dNTPs (e.g., dATP, dCTP, dGTP, and dTTP monomers). Additional embodiments further comprise a second primer, e.g., a second primer that is a hairpin oligonucleotide comprising a single-stranded region comprising an amplicon-specific priming segment; a double-stranded duplex region comprising a first self-complementary region hybridized to a second self-complementary region; a single-stranded loop region; and a blocker moiety.

Also described herein are embodiments of methods such as a method for producing a sequencing library. Exemplary methods relate to producing a sequencing library comprising an amplicon, the method comprising providing a reaction mixture comprising a hairpin oligonucleotide as described herein and a nucleic acid to be sequenced; and exposing the reaction mixture to conditions appropriate for producing an amplicon (e.g., an amplicon as described herein). In some embodiments, the reaction mixture comprises a polymerase comprising exonuclease activity. Embodiments of methods comprise monitoring a fluorescence signal at the emission wavelength of the fluorescent moiety (e.g., a real-time amplification method, e.g., a real-time PCR method, e.g., a real-time quantitative PCR method). In some embodiments, the methods comprise providing a second primer, wherein the second primer is a hairpin oligonucleotide comprising a single-stranded region comprising an amplicon-specific priming segment; a double-stranded duplex region comprising a first self-complementary region hybridized to a second self-complementary region; a single-stranded loop region; and a blocker moiety. Method embodiments relate to providing a sequencing library for input into a sequencing platform or system, e.g., for input into the workflow of a NGS system or platform. In some embodiments, the methods comprise sequencing the amplicon to produce a nucleotide sequence, wherein the nucleotide sequence comprises sequence from the nucleic acid and an index sequence (e.g., from a tag). Index sequences provide for multiplexing and demultiplexing capabilities useful for determining multiple sequences with more efficiency than existing technologies. Multiplex sequencing libraries comprise multiple nucleic acids, e.g., from multiple samples, subjects, alleles, etc. Accordingly, in some embodiments the methods comprise mixing a first amplicon and a second amplicon to produce a multiplex sequencing library. Accordingly, some embodiments further comprise associating a nucleotide sequence with a sample (e.g., demultiplexing). Additional embodiments comprise quantifying an amount of amplicon to provide in a sequencing library.

Accordingly, some embodiments relate to NGS sequencing libraries (e.g., produced according to embodiments of methods provided herein) for input into an NGS sequencing platform or system. Some embodiments relate to compositions comprising NGS sequencing libraries (e.g., produced according to embodiments of methods provided herein) for input into an NGS sequencing platform or system.

Some embodiments relate to a method for multiplex sequencing, the method comprising providing a first amplicon comprising a first nucleotide sequence comprising a first target sequence and a tag derived from a hairpin oligonucleotide, wherein the tag comprises a first index (index sequence); providing a second amplicon comprising a second nucleotide sequence comprising a second target sequence and a second tag derived from a hairpin oligonucleotide, wherein the second tag comprises a second index sequence; and mixing the first amplicon and the second amplicon to produce a multiplex sequencing library. Some embodiments of a method for multiplex sequencing comprise sequencing the multiplex sequencing library to produce a set of nucleotide sequences comprising a first nucleotide sequence and a second nucleotide sequence. Some embodiments for multiplex sequencing comprise demultiplexing the set of nucleotide sequences by assigning the first nucleotide sequence associated with the first index sequence to a first sample and assigning the second nucleotide sequence associated with the second index sequence to a second sample. Additional embodiments related to multiplex sequencing comprise sequencing a plurality of amplicons in a single reaction chamber to produce a plurality of nucleic acid sequences, wherein said amplicons are produced from two or more different samples; and identifying the sample from which each of said nucleic acid sequences is produced based on index sequences contained in each sequence of said plurality of nucleic acid sequences, wherein each index sequence is provided by a hairpin oligonucleotide as described herein.

Additional embodiments relate to a kit for generating a sequencing library comprising amplicons as described herein (e.g., amplicons as described herein, e.g., comprising a first portion comprising, derived from, and/or complementary to the target template and a second portion comprising a user-defined adaptor; e.g., amplicons comprising a nucleotide sequence derived from a target nucleic acid and a sequence derived from a hairpin oligonucleotide as described herein), the kit comprising a plurality of hairpin oligonucleotides as described herein, wherein each of said plurality of hairpin oligonucleotides comprises at least one of a plurality of index sequences; and a polymerase comprising exonuclease activity.

Further embodiments provide a system for generating nucleotide sequences, the system comprising a sequencing library comprising an amplicon, wherein said amplicon comprises a nucleotide sequence derived from a target nucleic acid and a sequence derived from a hairpin oligonucleotide as described herein; a thermocycler apparatus; and a computer for analyzing a nucleotide sequence and demultiplexing a plurality of nucleotide sequences. In some embodiments, systems comprise a fluorescence detector.

Some embodiments provide a hairpin oligonucleotide comprising a single-stranded region (e.g., comprising an amplicon-specific priming region and a tag); a double-stranded duplex region comprising a first self-complementary region hybridized to a second self-complementary region (e.g., with complete complementarity or comprising one or more (e.g., 1, 2, 3, 4, 4, 6, 7, 8, 9 10, or more) mismatches); a single-stranded loop region (e.g., comprising a PEG linker in some embodiments); a blocker moiety (e.g., a nuclease resistant moiety such as, e.g., a phosphorothioate or a peptide nucleic acid linkage, e.g., located near the junction of the single-stranded loop region and the double-stranded duplex region); a fluorescent moiety (e.g., xanthene, fluorescein, rhodamine, BODIPY, cyanine, coumarin, pyrene, phthalocyanine, FAM, JOE, Cy3, Cy5, Cy3.5, Cy5.5, TAMRA, ROX, HEX, or phycobiliprotein); and a quenching moiety (e.g., an Iowa Black Quencher or a Black Hole Quencher such as, e.g., BHQ-0, BHQ-1, BHQ-2, and BHQ-3), wherein the second self-complementary region comprises the fluorescent moiety and the quenching moiety, wherein the first self-complementary region and the second self-complementary region are not hybridized at or above a denaturing temperature in an amplification reaction, and wherein the first self-complementary region and the second self-complementary region are hybridized below a denaturing temperature in an amplification reaction.

Some embodiments provide a hairpin oligonucleotide comprising a single-stranded region (e.g., comprising an amplicon-specific priming region and a tag); a double-stranded duplex region comprising a first self-complementary region hybridized to a second self-complementary region (e.g., with complete complementarity or comprising one or more (e.g., 1, 2, 3, 4, 4, 6, 7, 8, 9 10, or more) mismatches); a single-stranded loop region (e.g., comprising a PEG linker in some embodiments); and a blocker moiety (e.g., a nuclease resistant moiety such as, e.g., a phosphorothioate or a peptide nucleic acid linkage, e.g., located near the junction of the single-stranded loop region and the double-stranded duplex region), wherein the first self-complementary region and the second self-complementary region are not hybridized at or above a denaturing temperature in an amplification reaction, and wherein the first self-complementary region and the second self-complementary region are hybridized below a denaturing temperature in an amplification reaction.

Some embodiments provide a hairpin oligonucleotide comprising a single-stranded region (e.g., comprising an amplicon-specific priming region); a double-stranded duplex region comprising a first self-complementary region hybridized to a second self-complementary region (e.g., with complete complementarity or comprising one or more (e.g., 1, 2, 3, 4, 4, 6, 7, 8, 9 10, or more) mismatches); a single-stranded loop region (e.g., comprising a PEG linker in some embodiments); a blocker moiety (e.g., a nuclease resistant moiety such as, e.g., a phosphorothioate or a peptide nucleic acid linkage, e.g., located near the junction of the single-stranded loop region and the double-stranded duplex region); a fluorescent moiety (e.g., xanthene, fluorescein, rhodamine, BODIPY, cyanine, coumarin, pyrene, phthalocyanine, FAM, JOE, Cy3, Cy5, Cy3.5, Cy5.5, TAMRA, ROX, HEX, or phycobiliprotein); and a quenching moiety (e.g., an Iowa Black Quencher or a Black Hole Quencher such as, e.g., BHQ-0, BHQ-1, BHQ-2, and BHQ-3), wherein the second self-complementary region comprises the fluorescent moiety and the quenching moiety, wherein the first self-complementary region and the second self-complementary region are not hybridized at or above a denaturing temperature in an amplification reaction, and wherein the first self-complementary region and the second self-complementary region are hybridized below a denaturing temperature in an amplification reaction.

Some embodiments provide a hairpin oligonucleotide comprising a single-stranded region (e.g., comprising an amplicon-specific priming region); a double-stranded duplex region comprising a first self-complementary region hybridized to a second self-complementary region (e.g., with complete complementarity or comprising one or more (e.g., 1, 2, 3, 4, 4, 6, 7, 8, 9 10, or more) mismatches); a single-stranded loop region (e.g., comprising a PEG linker in some embodiments); and a blocker moiety (e.g., a nuclease resistant moiety such as, e.g., a phosphorothioate or a peptide nucleic acid linkage, e.g., located near the junction of the single-stranded loop region and the double-stranded duplex region), wherein the first self-complementary region and the second self-complementary region are not hybridized at or above a denaturing temperature in an amplification reaction, and wherein the first self-complementary region and the second self-complementary region are hybridized below a denaturing temperature in an amplification reaction.

Some embodiments provide a hairpin oligonucleotide comprising a single-stranded region (e.g., comprising an amplicon-specific priming region and a tag); a double-stranded duplex region comprising a first self-complementary region hybridized to a second self-complementary region (e.g., with complete complementarity or comprising one or more (e.g., 1, 2, 3, 4, 4, 6, 7, 8, 9 10, or more) mismatches); and a PEG linker connecting the first self-complementary region and the second self-complementary region, wherein the first self-complementary region and the second self-complementary region are not hybridized at or above a denaturing temperature in an amplification reaction, and wherein the first self-complementary region and the second self-complementary region are hybridized below a denaturing temperature in an amplification reaction.

Some embodiments provide a hairpin oligonucleotide comprising a single-stranded region (e.g., comprising an amplicon-specific priming region); a double-stranded duplex region comprising a first self-complementary region hybridized to a second self-complementary region (e.g., with complete complementarity or comprising one or more (e.g., 1, 2, 3, 4, 4, 6, 7, 8, 9 10, or more) mismatches); and a PEG linker connecting the first self-complementary region and the second self-complementary region, wherein the first self-complementary region and the second self-complementary region are not hybridized at or above a denaturing temperature in an amplification reaction, and wherein the first self-complementary region and the second self-complementary region are hybridized below a denaturing temperature in an amplification reaction.

Additional embodiments relate to methods for sequencing a nucleic acid, the methods comprising providing a reaction mixture comprising one or more hairpin oligonucleotides as described herein, one or more nucleic acids to be sequenced, and a polymerase comprising exonuclease activity; exposing the reaction mixture to conditions appropriate for producing one or more amplicons; monitoring a fluorescence signal at the emission wavelength of the fluorescent moiety; quantifying one or more amounts or concentrations of one or more amplicons for provision in a sequencing library; sequencing the one or more amplicons to produce one or more nucleotide sequences, wherein each of the one or more nucleotide sequence comprises sequence from the nucleic acid and an index sequence; and associating each of the one or more nucleotide sequences with each of one or more samples (e.g., demultiplexing a set of nucleotide sequences comprising the one or more nucleotide sequences using the one or more index sequences).

The technology provided herein provides several advantages relative to existing technologies. First, some existing technologies use a hairpin primer in a first PCR reaction followed by a second PCR reaction in which a fusion primer primes off of the stem portion of the hairpin. In contrast to this approach in which two separate PCRs are needed to produce amplicons with flanking adaptor (e.g., comprising “universal”) sequences, the technology provided herein is based on a single amplification reaction to produce amplicons comprising adaptors that are compatible with NGS systems. Second, some existing technologies use hairpin primer variants designed only to produce DNA products with minimal side products for use as input template for a second PCR. In contrast, the technology described herein provides an oligonucleotide that has multiple functionalities to control fragment size; quantify and/or monitor amplification product; and/or to add adaptor sequences. These fundamental differences relative to the existing technologies ultimately lead to a significant improvement in the amount of hands-on time, over-all work flow time, and cost involved to produce a NGS amplicon library.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIGS. 1A-1F show-embodiments of hairpin primers according to the technology provided herein. FIG. 1A is a schematic drawing of one embodiment 100 of a hairpin primer comprising an amplicon-specific priming sequence 101, a tag 102, a single-stranded loop region 104, a fluorescent moiety 108, a quencher moiety 107, and a blocker (e.g., exonuclease resistant) moiety 106. FIG. 1B is a schematic drawing of a second embodiment 200 of a hairpin primer comprising an amplicon-specific priming sequence 201, a tag 202, a single-stranded loop region 204, and a blocker (e.g., nuclease resistant) moiety 206. FIG. 1C is a schematic drawing of one embodiment 110 of a hairpin primer comprising an amplicon-specific priming sequence 111, a single-stranded loop region 114, a fluorescent moiety 118, a quencher moiety 117, and a blocker (e.g., exonuclease resistant) moiety 116. FIG. 1D is a schematic drawing of one embodiment 210 of a hairpin primer comprising an amplicon-specific priming sequence 211, a single-stranded loop region 214, and a blocker (e.g., exonuclease resistant) moiety 216. FIG. 1E is a schematic drawing of one embodiment 220 of a hairpin primer comprising an amplicon-specific priming sequence 221, a tag 222, and a PEG linker 224. FIG. 1F is a schematic drawing of one embodiment 230 of a hairpin primer comprising an amplicon-specific priming sequence 231 and a PEG linker 234. White segments (both solid white fill and white fill with hatching) 103, 105, 203, 205, 113, 115, 213, 215, 223, 225, 233, and 235 represent components of double-stranded (duplex) elements (e.g., comprising the first self-complementary region and the second self-complementary region); black segments (both solid black fill and black fill with hatching) 101, 102, 104, 201, 202, 204, 111, 114, 211, 214, 221, 222, and 231 represent single-stranded elements; grey segments 224 and 234 represent PEG linkers. The adaptor sequence to be added to the nucleic acids of the library comprises 102, 103, and 104; 202, 203, and 204; 113 and 114; 213 and 214; 222 and 223; or 233.

FIGS. 2A-2C show multiple (three) different states of one embodiment of a hairpin primer 100. FIG. 2A shows an embodiment of a hairpin primer 100 at a denaturing temperature (e.g., a temperature greater than or equal to approximately 95° C.) at which the hairpin primer 100 is linear and does not comprise intra-molecular secondary structure; FIG. 2B shows an embodiment of a hairpin primer 100 at an intermediate temperature (e.g., a temperature of approximately 75° C.) at which intra-molecular secondary structure (e.g., the hairpin stem-loop comprising the double stranded element) forms; FIG. 2C shows an embodiment of a hairpin primer 100 at an annealing temperature (e.g., less than or equal to approximately 60° C.) at which the hairpin primer comprises intramolecular secondary structure and the amplicon-specific priming region 101 is hybridized to its complementary sequence on the target template 300.

FIG. 3 is a schematic showing stages of an embodiment of a nucleic acid amplification using one embodiment of a hairpin primer 100 comprising the fluorescent moiety (star). In FIG. 3, a hairpin primer 100 hybridizes to its complementary sequence on the target template 300, a polymerase (e.g., comprising 5′ to 3′ exonuclease activity) 400 (large grey circle) binds to the primed template (Step 1) and extends the 3′ end of the hairpin primer (e.g., from the amplicon-specific priming region) to form nucleic acid 500 comprising the fluorescent moiety in a quenched state (Step 2). Second strand synthesis by the polymerase produces nucleic acid 600 (Step 3). When the polymerase encounters the 5′ end of the double-stranded (e.g., hairpin) region of the nucleic acid 500, the exonuclease activity of the polymerase degrades the double-stranded structure from the 5′ end of the hairpin, releasing the fluorescent moiety (star) and the quenching moiety (pentagon) (Step 4). Separation in space of the fluorescent moiety 108 and the quenching moiety 107 (e.g., as the fluorescent moiety and the quenching moiety diffuse away from one another in the reaction mixture) allows the fluorescent moiety 108 to fluoresce (multiply outlined (e.g., “shining”) star). Degradation of the duplex region by the exonuclease of the polymerase is blocked by the blocker (exonuclease resistant) moiety (small dark circle) at a defined location, leaving a defined end. Degradation of the duplex region exposes the adaptor sequence (hatched region) and the polymerase continues synthesis to the end of the template, which is delimited by the blocker (e.g., nuclease resistant) moiety (Step 5). The resulting amplicon comprises target sequence (black filled segment) and adaptor sequence (black filled region with hatching).

FIGS. 4A-4D show the results of modeling hairpin primer structure using software (UNAfold, Rensselaer Polytechnic Institute). The predicted structures and free energies of hairpin formation at 70° C., 62° C., and 55° C. are provided for the primers F_egfr_trP1 (FIG. 4A), R_egfr_b1_A (FIG. 4B), F_Chr1_trP1 (FIG. 4C), and R_Chr1_b1_A (FIG. 4D).

FIGS. 5A-5B show plots from real-time amplification reactions using the primers F_egfr_trP1, R_egfr_b1_A, F_Chr1_trP1, and R_Chr1_b1_A (see Table 1) and probes (see Table 3) in a two-plex amplification of EGFR (FIG. 5A) and chromosome 1 (FIG. 5B) targets. The plots show the accumulation of product in arbitrary units (Rn) as a function of cycle number.

FIGS. 6A-6B show the measured sizes of amplification products (FIG. 6A) and predicted structures of amplification products (FIG. 6B) for an amplification reaction using the primers F_egfr_trP1, R_egfr_b1_A, F_Chr1_trP1, and R_Chr1_b1_A (see Table 1) in a two-plex amplification of EGFR and chromosome 1 targets. FIG. 6A is a plot showing the experimentally measured relative amounts of amplification products over a range of sizes from approximately 5 to 500 base pairs. FIG. 6B is a schematic showing the predicted structures of exemplary (e.g., predominant) intermediate products and/or end point products of the amplification reaction using the primers F_egfr_trP1, R_egfr_b1_A, F_Chr1_trP1, and R_Chr1_LA (see Table 1) in a two-plex amplification of EGFR and chromosome 1 targets. The fluorescent moiety, quencher moiety, and blocker (e.g., exonuclease resistant) moiety are shown in FIG. 6B as a star, pentagon, and circle, respectively. Roman numerals are used to label various predicted products of the amplification.

FIGS. 7A-7B show the measured sizes of amplification products after enzymatic treatment with lambda exonuclease and Klenow DNA polymerase (FIG. 7A) and predicted structures of amplification products after treatment with lambda exonuclease and Klenow DNA polymerase (FIG. 7B) for an amplification reaction using the primers F_egfr_trP1, R_egfr_b1_A, F_Chr1_trP1, and R_Chr1_b1_A (see Table 1) in a two-plex amplification of EGFR and chromosome 1 targets. FIG. 7A is a plot showing the experimentally measured relative amounts of amplification products after treatment with lambda exonuclease and Klenow DNA polymerase over a range of sizes from approximately 5 to 300 bp. FIG. 7B is a schematic showing the predicted structure of an exemplary amplification product after treatment with lambda exonuclease and Klenow DNA polymerase. The blocker (e.g., exonuclease resistant) moiety is shown in FIG. 7B as a circle.

FIG. 8 is a plot showing the mapping efficiencies for sequences generated using standard fusion primers (“Run 1”, “Run 2”, “Run 3”, and “Run 4”), using standard adaptor ligation to a fragmented library (“Run 5”), and using the hairpin primer technology as provided herein (“Run 6”, “Run 7”, and “Run 8”). Total reads (triangles and line plot) and the percentages of the total reads that could be mapped (black portion of each column and percentage indicated by the lower number on each column) and unmapped (lighter (grey) portion of each column and percentage indicated by the upper number on each column) are shown for 8 sequencing runs using these technologies.

FIG. 9 is a flowchart showing an exemplary embodiment of method for preparing amplicon libraries and sequencing. OS-primer refers to a “one-step primer”, e.g., a hairpin primer as provided herein.

FIG. 10 is a plot showing the mapping efficiencies for sequencing according to embodiments of the technology provided herein. Column 1 shows mapped and unmapped reads for both Run 1 and Run 2 of sample B1-356, column 2 shows mapped and unmapped reads for both Run 1 and Run 2 of sample B3-384, column 3 shows mapped and unmapped reads for both Run 1 and Run 2 of sample B1-356, and column 4 shows mapped and unmapped reads for both Run 1 and Run 2 for sample B3-384.

FIG. 11 is a plot showing the mapped EGFR sequencing reads (left black bar of each pair of bars) and chromosome 1 sequencing reads (right diagonally hatched bar of each pair of bars) based on assigning reads to samples using barcodes (e.g., barcode B1 or barcode B3). Specific sequence reads from EGFR or from chromosome 1 were counted and normalized to assess relative copy number status of EGFR compared to the copy number of chromosome 1, which served as a control. FIG. 11 also shows the relative copy number of EGFR and chromosome 1 based on using sequence count data from sample 356 as a reference and a normalized EGFR copy number for sample 384.

FIGS. 12A-12B show embodiments of the technology comprising a PEG linker. FIG. 12A shows the structures of embodiments of hairpin oligonucleotides having similar structures, but one having a PEG loop (lower oligonucleotide “OS-s-primer (PEG loop)”) and one having conventional nucleotides and phosphorothioate linkages (“*”) (upper oligonucleotide “OS-primer (DNA loop)”). FIG. 12B shows the structure of a PEG linker comprising n repeating units, wherein n equals 1 to 40, e.g., n equals 1, 2, 3, 4, 5, 6, 7, 8, 9, 10, 11, 12, 13, 14, 15 16, 17, 18, 19, 20, 21, 22, 23, 24, 25, 26, 27, 28, 29, 30, 31, 32, 33, 34, 35, 36, 37, 38, 39, or 40.

FIG. 13 is a plot showing the amplicon quantity in picograms for amplification reactions using the hairpin oligonucleotides depicted in FIG. 12. The left column shows the amplicon quantity for an amplification reaction using a hairpin oligonucleotides having conventional nucleotides and phosphorothioate linkages (“*”) (“OS-primer”). The right column shows the amplicon quantity for an amplification reaction using a hairpin oligonucleotide having a PEG loop (“OS-s-primer”).

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to the manipulation and characterization of nucleic acids and particularly, but not exclusively, to methods and compositions relating to oligonucleotide primers and probes for amplifying, quantifying, and sequencing nucleic acids.

In this detailed description of the various embodiments, section headings used herein are for organizational purposes only and are not to be construed as limiting the described subject matter in any way. For purposes of explanation, numerous specific details are set forth to provide a thorough understanding of the embodiments disclosed. One skilled in the art will appreciate that the various embodiments described herein may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, a “nucleic acid” shall mean any nucleic acid molecule, including, without limitation, DNA, RNA, and hybrids thereof. The nucleic acid bases that form nucleic acid molecules can be the bases A, C, G, T and U, as well as derivatives thereof. Derivatives of these bases are well known in the art. The term should be understood to include, as equivalents, analogs of either DNA or RNA made from nucleotide analogs. The term as used herein also encompasses cDNA, that is complementary, or copy, DNA produced from an RNA template, for example, by the action of a reverse transcriptase.

As used herein, “nucleic acid sequencing data”, “nucleic acid sequencing information”, “nucleic acid sequence”, “genomic sequence”, “genetic sequence”, “fragment sequence”, or “nucleic acid sequencing read” denotes any information or data that is indicative of the order of the nucleotide bases (e.g., adenine, guanine, cytosine, and thymine/uracil) in a molecule (e.g., a whole genome, a whole transcriptome, an exome, oligonucleotide, polynucleotide, fragment, etc.) of DNA or RNA.

It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

Reference to a base, a nucleotide, or to another molecule may be in the singular or plural. That is, “a base” may refer to a single molecule of that base or to a plurality of the base, e.g., in a solution.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually, oligonucleotides range in size from a few monomeric units, e.g. 3-4, to several hundreds of monomeric units. Whenever a polynucleotide such as an oligonucleotide is represented by a sequence of letters, such as “ATGCCTG”, it will be understood that the nucleotides are in 5′ to 3′ order from left to right and that “A” or “a” denotes deoxyadenosine, “C” or “c” denotes deoxycytidine, “G” or “g” denotes deoxyguanosine, and “T” or “t” denotes thymidine, unless otherwise noted. The letters A, C, G, and T may be used to refer to the bases themselves, to nucleosides, or to nucleotides comprising the bases, as is standard in the art.

In some embodiments, nucleic acids comprise a universal or modified base such as deoxyinosine, inosine, 7-deaza-2′-deoxyinosine, 2-aza-2′-deoxyinosine, 2′-O-Me inosine, 2′-F inosine, deoxy 3-nitropyrrole, 3-nitropyrrole, 2′-O-Me 3-nitropyrrole, 2′-F 3-nitropyrrole, 1-(2′-deoxy-beta-D-ribofuranosyl)-3-nitropyrrole, deoxy 5-nitroindole, 5-nitroindole, 2′-O-Me 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-nitroindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′-O-methoxyethyl nebularine, 2′-O-methoxyethyl 5-nitroindole, 2′-O-methoxyethyl 4-nitro-benzimidazole, 2′-O-methoxyethyl 3-nitropyrrole, and combinations thereof.

As used herein, the term “target nucleic acid” or “target nucleotide sequence” refers to any nucleotide sequence (e.g., RNA or DNA), the manipulation of which may be deemed desirable for any reason by one of ordinary skill in the art. In some embodiments, “target nucleic acid” refers to a nucleotide sequence whose nucleotide sequence is to be determined or is desired to be determined. In some embodiments, the term “target nucleotide sequence” refers to a sequence to which a partially or completely complementary primer or probe is generated.

As used herein, the term “region of interest” refers to a nucleic acid that is analyzed (e.g., using one of the compositions, systems, or methods described herein). In some embodiments, the region of interest is a portion of a genome or region of genomic DNA (e.g., comprising one or chromosomes or one or more genes). In some embodiments, mRNA expressed from a region of interest is analyzed.

As used herein, the term “corresponds to” or “corresponding” is used in reference to a contiguous nucleic acid or nucleotide sequence (e.g., a subsequence) that is complementary to, and thus “corresponds to”, all or a portion of a target nucleic acid sequence.

As used herein, “complementary” generally refers to specific nucleotide duplexing to form canonical Watson-Crick base pairs, as is understood by those skilled in the art. However, complementary also includes base-pairing of nucleotide analogs that are capable of universal base-pairing with A, T, G or C nucleotides and locked nucleic acids that enhance the thermal stability of duplexes. One skilled in the art will recognize that hybridization stringency is a determinant in the degree of match or mismatch in the duplex formed by hybridization.

As used herein, “moiety” refers to one of two or more parts into which something may be divided, such as, for example, the various parts of an oligonucleotide, a molecule, a chemical group, a domain, a probe, etc.

As used herein, the term “library” refers to a plurality of nucleic acids, e.g., a plurality of different nucleic acids. In some embodiments, a “library” is a “library panel” or an “amplicon library panel”. As used herein, an “amplicon library panel” is a collection of amplicons that are related, e.g., to a disease (e.g., a polygenic disease), disease progression, developmental defect, constitutional disease (e.g., a state having an etiology that depends on genetic factors, e.g., a heritable (non-neoplastic) abnormality or disease), metabolic pathway, pharmacogenomic characterization, trait, organism (e.g., for species identification), group of organisms, geographic location, organ, tissue, sample, environment (e.g., for metagenomic and/or ribosomal RNA (e.g., ribosomal small subunit (SSU), ribosomal large subunit (LSU), 5S, 16S, 18S, 23S, 28S, internal transcribed sequence (ITS) rRNA) studies), gene, chromosome, etc. For example, a cancer amplicon panel may comprise a collection of amplicons comprising hundreds, thousands, or more loci, regions, genes, single nucleotide polymorphisms, alleles, markers, etc. that are associated with cancer. In some embodiments, an amplicon library panel provides for highly multiplexed and targeted resequencing, e.g., to detect mutations associated with disease. In some embodiments, a “library” comprises a plurality (e.g., collection) of “library fragments”; a “library fragment” is a nucleic acid. In some embodiments, library fragments are produced by fragmenting a larger nucleic acid, e.g., by physical (e.g., shearing), enzymatic (e.g., by nuclease), and/or chemical treatment. In some embodiments, library fragments are produced by amplification (e.g., PCR) and are thus amplicons corresponding to and/or derived from a nucleic acid (e.g., a nucleic acid to be sequenced).

For example, embodiments of a cancer panel comprise specific genes or mutations in genes that have established relevancy to a particular cancer phenotype (e.g., one or more of ABL1, AKT1, AKT2, ATM, PDGFRA, EGFR, FGFR (e.g., FGFR1, FGFR2, FGFR3), BRAF (e.g., comprising a mutation at V600, e.g., a V600E mutation), RUNX1, TET2, CBL, EGFR, FLT3, JAK2, JAK3, KIT, RAS (e.g., KRAS (e.g., comprising a mutation at G12, G13, or A146, e.g., a G12A, G12S, G12C, G12D, G13D, or A146T mutation), HRAS (e.g., comprising a mutation at G12, e.g., a G12V mutation), NRAS (e.g., comprising a mutation at Q61, e.g., a Q61R or Q61K mutation)), MET, PIK3CA (e.g., comprising a mutation at H1047, e.g., a H1047L, H1047L, or H1047R mutation), PTEN, TP53 (e.g., comprising a mutation at R248, Y126, G245, or A159, e.g., a R248W, G245S, or A159D mutation), VEGFA, BRCA, RET, PTPN11, HNHF1A, RB1, CDH1, ERBB2, ERBB4, SMAD4, SKT11 (e.g., comprising a mutation at Q37), ALK, IDH1, IDH2, SRC, GNAS, SMARCB1, VHL, MLH1, CTNNB1, KDR, FBXW7, APC, CSF1R, NPM1, MPL, SMO, CDKN2A, NOTCH1, CDK4, CEBPA, CREBBP, DNMT3A, FES, FOXL2, GATA1, GNAll, GNAQ, HIF1A, IKBKB, MEN1, NF2, PAX5, PIK3R1, PTCH1, STK11, etc.). Some amplicon panels are directed toward particular “cancer hotspots”, that is, regions of the genome containing known mutations that correlate with cancer progression and therapeutic resistance.

In some embodiments, an amplicon panel for a single gene includes amplicons for the exons of the gene (e.g., 1, 2, 3, 4, 5, 6, 7, 8, 9, 10, 11, 12, 13, 14, 15, 16, 17, 18, 19, 20, or more exons). In some embodiments, an amplicon panel for species (or strain, sub-species, type, sub-type, genus, or other taxonomic level and/or operational taxonomic unit (OTU) based on a measure of phylogenetic distance) identification may include amplicons corresponding to a suite of genes or loci that collectively provide a specific identification of one or more species (or strain, sub-species, type, sub-type, genus, or other taxonomic level) relative to other species (or strain, sub-species, type, sub-type, genus, or other taxonomic level) (e.g., for bacteria (e.g., MRSA), viruses (e.g., HIV, HCV, HBV, respiratory viruses, etc.)) or that are used to determine drug resistance(s) and/or sensitivity/ies (e.g., for bacteria (e.g., MRSA), viruses (e.g., HIV, HCV, HBV, respiratory viruses, etc.)).

The amplicons of the panel typically comprise 100 to 1000 base pairs, e.g., in some embodiments the amplicons of the panel comprise approximately 100, 125, 150, 175, 200, 225, 250, 275, 300, 325, 350, 375, 400, 325, 350, 375, 400, 425, 450, 475, 500, 525, 550, 575, 600, 625, 650, 675, 700, 725, 750, 775, 800, 825, 850, 875, 900, 925, 950, 975, or 1000 base pairs. In some embodiments, an amplicon panel comprises a collection of amplicons that span a genome, e.g., to provide a genome sequence.

The amplicon panel is often produced through use of amplification oligonucleotides (e.g., to produce the amplicon panel from the sample) and/or oligonucleotide probes for sequencing disease-related genes, e.g., to assess the presence of particular mutations and/or alleles in the genome. In some embodiments, 10, 20, 30, 40, 50, 60, 70, 80, 90, 100, 150, 200, 300, 400, 500, 1000, or more genes, loci, regions, etc. are targeted to produce, e.g., 10, 20, 30, 40, 50, 60, 70, 80, 90, 100, 150, 200, 300, 400, 500, 1000, or more amplicons. In some embodiments, the amplicons are produced in a highly multiplexed, single tube amplification reaction. In some embodiments, the amplicons are produced in a collection of singleplex amplification reactions (e.g., 10 to 100, 100 to 1000, or 1000 or more reactions). In some embodiments, the multiple singleplex amplification reactions are pooled. In some embodiments, the singleplex amplification reactions are performed in parallel.

As used herein, a “subsequence” of a nucleotide sequence refers to any nucleotide sequence contained within the nucleotide sequence, including any subsequence having a size of a single base up to a subsequence that is one base shorter than the nucleotide sequence.

The phrase “sequencing run” refers to any step or portion of a sequencing experiment performed to determine some information relating to at least one biomolecule (e.g., nucleic acid molecule).

As used herein, the phrase “dNTP” means deoxynucleotidetriphosphate, where the nucleotide comprises a nucleotide base, such as A, T, C, G or U.

The term “monomer” as used herein means any compound that can be incorporated into a growing molecular chain by a given polymerase. Such monomers include, without limitation, naturally occurring nucleotides (e.g., ATP, GTP, TTP, UTP, CTP, dATP, dGTP, dTTP, dUTP, dCTP, synthetic analogs), precursors for each nucleotide, non-naturally occurring nucleotides and their precursors or any other molecule that can be incorporated into a growing polymer chain by a given polymerase.

A “polymerase” is an enzyme generally for joining 3′-OH 5′-triphosphate nucleotides, oligomers, and their analogs. Polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase 1, Klenow fragment, Thermophilus aquaticus (Taq) DNA polymerase, Thermus thermophilus (Tth) DNA polymerase, Vent DNA polymerase (New England Biolabs), Deep Vent DNA polymerase (New England Biolabs), Bacillus stearothermophilus (Bst) DNA polymerase, DNA Polymerase Large Fragment, Stoeffel Fragment, 9° N DNA Polymerase, 9° Nm polymerase, Pyrococcus furiosis (Pfu) DNA Polymerase, Thermus filiformis (Tfl) DNA Polymerase, RepliPHI Phi29 Polymerase, Thermococcus litoralis (Tli) DNA polymerase, eukaryotic DNA polymerase beta, telomerase, Therminator polymerase (New England Biolabs), KOD HiFi. DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting and/or molecular evolution, and polymerases cited in U.S. Pat. Appl. Pub. No. 2007/0048748 and in U.S. Pat. Nos. 6,329,178; 6,602,695; and 6,395,524. These polymerases include wild-type, mutant isoforms, and genetically engineered variants such as exo-polymerases; polymerases with minimized, undetectable, and/or decreased 3′→5′ proofreading exonuclease activity, and other mutants, e.g., that tolerate labeled nucleotides and incorporate them into a strand of nucleic acid. In some embodiments, the polymerase is designed for use, e.g., in real-time PCR, high fidelity PCR, next-generation DNA sequencing, fast PCR, hot start PCR, crude sample PCR, robust PCR, and/or molecular diagnostics. Such enzymes are available from many commercial suppliers, e.g., Kapa Enzymes, Finnzymes, Promega, Invitrogen, Life Technologies, Thermo Scientific, Qiagen, Roche, etc. In some embodiments, the polymerase has 5′→>3′ exonuclease activity and can thus degrade a nucleic acid from a 5′ end in addition to catalyzing synthesis of a nucleic acid from a 3′-OH of a nucleic acid (e.g., from a primer, e.g., a hairpin primer). In some embodiments the polymeras (e.g., a high-fidelity polymerase) comprises a proof-reading activity, a 3′ exonuclease activity, and/or a strand displacement activity, but lacks a 5′ exonuclease activity.

The term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method. As used herein, the single stranded (e.g., amplicon-specific) portion of a hairpin primer may serve to prime the synthesis of a nucleic acid.

The term “annealing” or “priming” as used herein refers to the apposition of an oligodeoxynucleotide or nucleic acid to a template nucleic acid, whereby the apposition enables the polymerase to polymerize nucleotides into a nucleic acid molecule that is complementary to the template nucleic acid or a portion thereof. The term “hybridizing” as used herein refers to the formation of a double-stranded nucleic acid from complementary single stranded nucleic acids. There is no intended distinction between the terms “annealing” and “hybridizing”, and these terms will be used interchangeably. The sequences of primers may comprise some mismatches, so long as they can be hybridized with templates and serve as primers. The term “substantially complementary” is used herein to signify that the primer is sufficiently complementary to hybridize selectively to a template nucleic acid sequence under the designated annealing conditions or stringent conditions, such that the annealed primer can be extended by a polymerase to form a complementary copy of the template.

As used herein, a “system” denotes a set of components, real or abstract, comprising a whole where each component interacts with or is related to at least one other component within the whole. Various nucleic acid sequencing platforms, nucleic acid assembly, and/or nucleic acid sequence mapping systems (e.g., computer software and/or hardware) are described, e.g., in U.S. Pat. Appl. Pub. No. 2011/0270533, which is incorporated herein by reference in its entirety.

As used herein the term “isolating” is intended to mean that the material in question exists in a physical milieu distinct from that in which it occurs in nature and/or it has been completely or partially separated, isolated, or purified from other nucleic acid molecules.

As used herein, an “index” shall generally mean a distinctive or identifying mark or characteristic, e.g., a virtual or a known nucleotide sequence that is used for marking a DNA fragment (e.g., an amplicon) and/or a library (e.g., an amplicon library) and for constructing a multiplex library. A library includes, but is not limited to, a genomic DNA library, a cDNA library, an amplicon library, and a ChIP library. A plurality of DNAs, each of which is separately marked with an index, may be pooled together to form a multiplex indexed library for performing sequencing simultaneously, in which each index is sequenced together with flanking DNA in the same construct and thereby serves as an index for the DNA fragment and/or library marked by the index. In some embodiments, an index is made with a specific nucleotide sequence having 1, 2, 3, 4, 5, 6, 7, 8, 9, 10, or more nucleotides in length. The length of an index may be increased along with the maximum sequencing length of a sequencer. The term index is interchangeable with the terms “barcode” and “barcode sequence”.

As used herein, “virtual” shall generally mean not in actual form but existing or resulting in effect.

As used herein, “restriction enzyme recognition site” and “restriction enzyme binding site” are interchangeable.

The term “sample” is used in its broadest sense. In one sense it can refer to an animal cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

As used herein, “multiplex” refers to using multiple amplification primers (e.g., multiple hairpin oligonucleotides, e.g., wherein each hairpin oligonucleotide comprises a different tag or index sequence) to amplify the same pool of nucleic acids. “Multiplex sequencing”, as used herein, refers to pooling multiple amplicons (e.g., from multiple subjects, samples, etc.) and sequencing the pool in a single sequencing run.

As used herein, “demultiplexing” refers to assigning a nucleotide sequence to a subject or sample and “demultiplexed” refers to a nucleotide sequence that has been assigned to a subject or sample. For example, in multiplexed sequencing each amplicon comprises an index that corresponds to the subject or sample from which the nucleic acid producing the amplicon was isolated or derived. After multiple amplicons are mixed together and sequenced, the index is used to identify the nucleotide sequence that belongs to each subject or sample.

As used herein, an “n-plex” detection (e.g., two-plex, three-plex, four-plex, etc.) is a detection in which n (e.g., 2, 3, 4, etc.) targets are detected (e.g., in some embodiments simultaneously) in the same detection reaction (e.g., an amplification reaction, e.g., a polymerase chain reaction). Accordingly, as used herein, a “plexed” detection, assay, etc. is one in which multiple analytes, targets, etc. are assayed in one reaction.

### Description

The technology generally relates to oligonucleotides and methods of using “hairpin” or “stem-loop” oligonucleotides to produce a nucleic acid library for next-generation sequencing.

In general, the technology provides an oligonucleotide comprising a double-stranded (e.g., duplex) section that forms by intra-molecular folding and a single-stranded section. The single-stranded section is free to hybridize to a complementary sequence of another nucleic acid (e.g., a target template), where the oligonucleotide acts as a primer in an amplification reaction (e.g., a polymerase chain reaction) to produce amplicons. The resulting amplicons comprise a first portion corresponding to (e.g., comprising, derived from, and/or complementary to) the target template and a second portion comprising a sequence provided by the hairpin primers (e.g., an adaptor, e.g., an adaptor comprising a tag). Modification of specific nucleotides or chemical bonds between nucleotides (e.g., such as incorporating a nuclease resistant moiety (e.g., a phosphorothioate bond and/or a PEG linker)) in the oligonucleotides provides precise control of the size and content (e.g., sequence) of the adaptor sequence at ends of the amplicons. Furthermore, in some embodiments the hairpin oligonucleotides comprise a fluorescent moiety and, in some embodiments, a quenching moiety, which provides for the monitoring and/or quantitation of amplicon generation through fluorescence measurements (e.g., by a real-time quantitative amplification reaction (e.g., PCR)).

The technology provides an efficient “one-step/one-tube” generation and quantification of an amplicon library for NGS. In particular, these advantages are related to new primer designs having the following unique combination of components: First, the NGS platform-dependent adaptor (e.g., “universal”) sequences are kept “hidden” by the stem-loop structure during key PCR temperature ranges, thus minimizing or eliminating complex hybridization between various templates and primers. As a result, off-target amplicon formation is minimized or eliminated, which ultimately increases the efficiency of PCR (e.g., multiplex PCR) with minimal side products. Second, the “blocker” nuclease-resistant moiety (e.g., a phosphorothioate bond) is placed at a strategic location within the primer to control the extent of primer hydrolysis by the polymerase nuclease activity, thus producing products with defined ends. Third, fluorescent and quenching moieties attached at appropriate locations provide amplification product monitoring and quantification during amplification. As such, the present technology provides robust single-tube production of multi-amplicon libraries ready for input into a NGS system with minimal hands-on time, facile integration into automated workflows, and significant decrease in overall work-flow time and cost.

### Hairpin Oligonucleotides

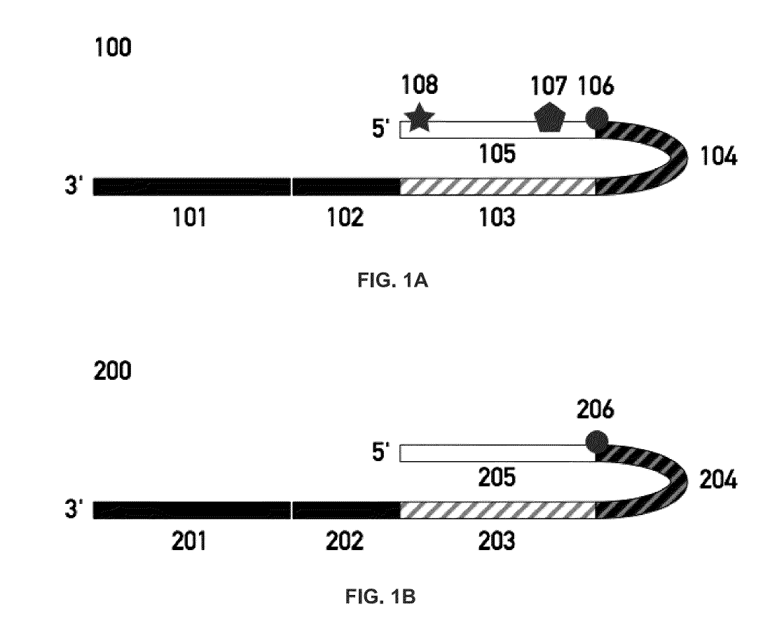

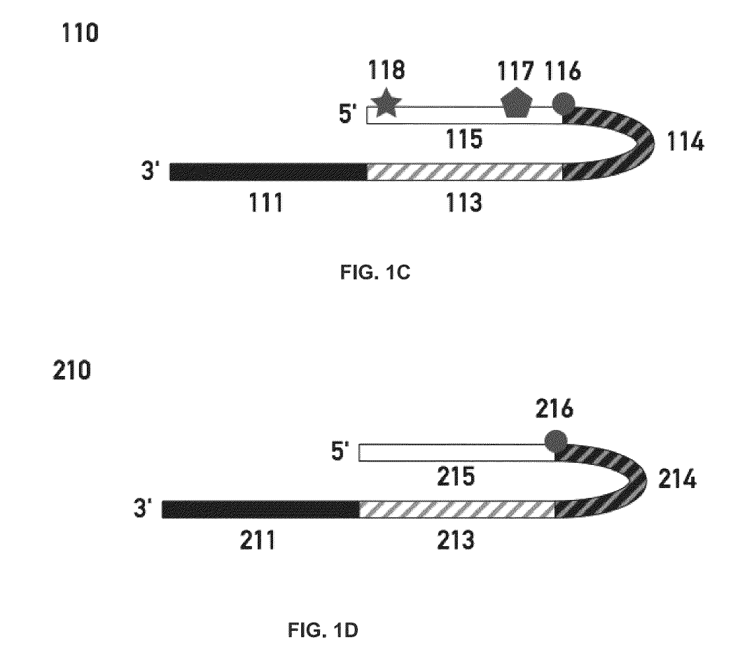

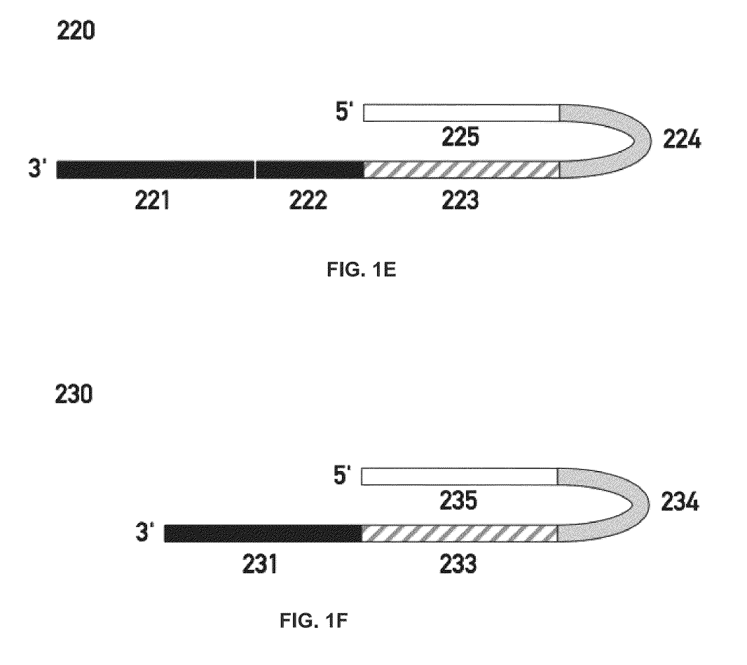

In some embodiments, the technology provides hairpin (e.g., “stem-loop”) oligonucleotides (see, e.g., FIG. 1). In some embodiments, the hairpin oligonucleotides comprise fluorescence and quencher moieties (see, e.g., FIG. 1A and FIG. 1C). In some embodiments, the hairpin oligonucleotides do not comprise fluorescence and quencher moieties (see, e.g., FIG. 1B, FIG. 1D, FIG. 1E, and FIG. 1F).

For example, an embodiment of the hairpin oligonucleotide 100 comprises a single-stranded region (e.g., black segments 101 and 102), a double-stranded duplex region (e.g., white hatched segment 103 hybridized to complementary white filled segment 105), and a single-stranded loop region (e.g., black hatched segment 104). Additionally, in some embodiments, the oligonucleotide 100 comprises several segments, including a first portion comprising, derived from, and/or complementary to the target template (e.g., an amplicon-specific priming segment) 101, a tag 102, a first self-complementary region 103, a single-stranded loop region 104, a second self-complementary region 105, a blocker (e.g., nuclease-resistant (e.g., exonuclease-resistant (e.g., 5′ to 3′ exonuclease-resistant))) moiety 106, a quencher moiety 107, and a fluorescent moiety 108 (FIG. 1A).

Another embodiment of the hairpin oligonucleotide 200 comprises a single-stranded region (e.g., black segments 201 and 202), a double-stranded duplex region (e.g., white hatched segment 203 hybridized to complementary white filled segment 205), and a single-stranded loop region (e.g., black hatched segment 204). Additionally, in some embodiments, the oligonucleotide 200 comprises several segments, including a first portion comprising, derived from, and/or complementary to the target template (e.g., an amplicon-specific priming segment) 201, a tag 202, a first self-complementary region 203, a single-stranded loop region 204, a second self-complementary region 205, and a blocker (e.g., nuclease-resistant (e.g., exonuclease-resistant (e.g., 5′ to 3′ exonuclease-resistant))) moiety 206 (FIG. 1B).

A third embodiment of the hairpin oligonucleotide 110 comprises a single-stranded region (e.g., black segment 111), a double-stranded duplex region (e.g., white hatched segment 113 hybridized to complementary white filled segment 115), and a single-stranded loop region (e.g., black hatched segment 114). Additionally, in some embodiments, the oligonucleotide 110 comprises several segments, including a first portion comprising, derived from, and/or complementary to the target template (e.g., an amplicon-specific priming segment) 111, a first self-complementary region 113, a single-stranded loop region 114, a second self-complementary region 115, a blocker (e.g., nuclease-resistant (e.g., exonuclease-resistant (e.g., 5′ to 3′ exonuclease-resistant))) moiety 116, a quencher moiety 117, and a fluorescent moiety 118 (FIG. 1C).

A fourth embodiment of the hairpin oligonucleotide 210 comprises a single-stranded region (e.g., black segment 211), a double-stranded duplex region (e.g., white hatched segment 213 hybridized to complementary white filled segment 215), and a single-stranded loop region (e.g., black hatched segment 214). Additionally, in some embodiments, the oligonucleotide 210 comprises several segments, including a first portion comprising, derived from, and/or complementary to the target template (e.g., an amplicon-specific priming segment) 211, a first self-complementary region 213, a single-stranded loop region 214, a second self-complementary region 215, and a blocker (e.g., nuclease-resistant (e.g., exonuclease-resistant (e.g., 5′ to 3′ exonuclease-resistant))) moiety 216 (FIG. 1B).

A fifth embodiment of the hairpin oligonucleotide 220 comprises a single-stranded region (e.g., black segment 221), a tag 222, a double-stranded duplex region (e.g., white hatched segment 223 hybridized to complementary white filled segment 225), and a PEG linker (e.g., grey segment 224). Additionally, in some embodiments, the oligonucleotide 220 comprises several segments, including a first portion comprising, derived from, and/or complementary to the target template (e.g., an amplicon-specific priming segment) 221, a first self-complementary region 223, a PEG linker 224, and a second self-complementary region 225 (FIG. 1E).

A sixth embodiment of the hairpin oligonucleotide 230 comprises a single-stranded region (e.g., black segment 231), a double-stranded duplex region (e.g., white hatched segment 233 hybridized to complementary white filled segment 235), and a PEG linker (e.g., grey segment 234). Additionally, in some embodiments, the oligonucleotide 230 comprises several segments, including a first portion comprising, derived from, and/or complementary to the target template (e.g., an amplicon-specific priming segment) 231, a first self-complementary region 233, a PEG linker 234, and a second self-complementary region 235 (FIG. 1F).

While the description refers to particular exemplary embodiments of the oligonucleotides (e.g., 100 and 200) to describe the relationships, functions, structures, etc. of the various components and segments, one of ordinary skill in the art understands that concepts relating to the structure and function of these exemplary embodiments are equally applicable to the other embodiments. For example, one of ordinary skill in the art understands that discussion of the first self-complementary region 103 and the second self-complementary region 105 in the embodiment represented in FIG. 1 as 100 applies also to the first self-complementary region and the second self-complementary region in other embodiments and thus one of ordinary skill in the art understands that the various segments and features described in the various embodiments are regarded to be equivalent. The same applies to single-stranded regions; double-stranded regions; portions comprising, derived from, and/or complementary to a target template (e.g., an amplicon-specific priming segment); tags; adaptors; and other components and segments described herein.

Thus, hairpin oligonucleotide 110 (FIG. 1C) is similar in structure and function as hairpin oligonucleotide 100 (FIG. 1A), though hairpin oligonucleotide 110 lacks a tag (e.g., hairpin oligonucleotide 110 is tagless). Likewise, hairpin oligonucleotide 210 (FIG. 1D) is similar in structure and function as hairpin oligonucleotide 200 (FIG. 1B), though hairpin oligonucleotide 210 lacks a tag (e.g., hairpin oligonucleotide 210 is tagless). Hairpin oligonucleotide 220 (FIG. 1E) is similar in structure and function as hairpin oligonucleotide 200 (FIG. 1B), though hairpin oligonucleotide 220 lacks a blocker (hairpin oligonucleotide 220 is blockerless) and has a PEG linker instead of a single-stranded loop segment. Hairpin oligonucleotide 230 (FIG. 1F) is similar in structure and function as hairpin oligonucleotide 220 (FIG. 1E), though hairpin oligonucleotide 230 lacks a tag (hairpin oligonucleotide 230 is tagless). Or, alternatively, hairpin oligonucleotide 230 (FIG. 1F) is similar in structure and function as hairpin oligonucleotide 210 (FIG. 1E), though hairpin oligonucleotide 230 lacks a blocker (hairpin oligonucleotide 230 is blockerless) and has a PEG linker instead of a single-stranded loop segment.

Accordingly, in exemplary embodiments the hairpin oligonucleotide comprises a first portion 101 comprising, derived from, and/or complementary to the target template (e.g., an amplicon-specific priming segment); and a second portion comprising a user-defined adaptor (e.g., an adaptor comprising a tag 102 (e.g., a tag comprising a linker, index, capture sequence, restriction site, primer binding site, antigen, and/or other functional site) and/or comprising a universal sequence (e.g., comprising a platform-dependent sequence)).

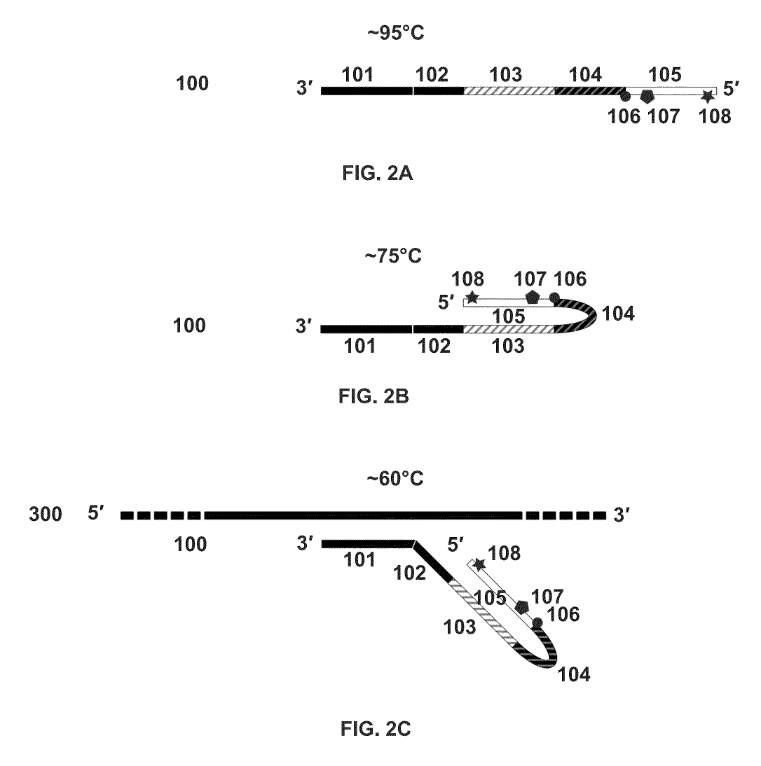

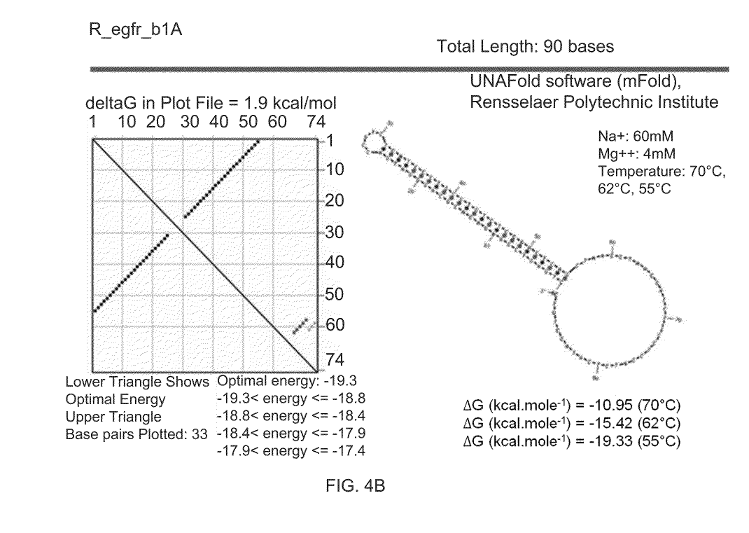

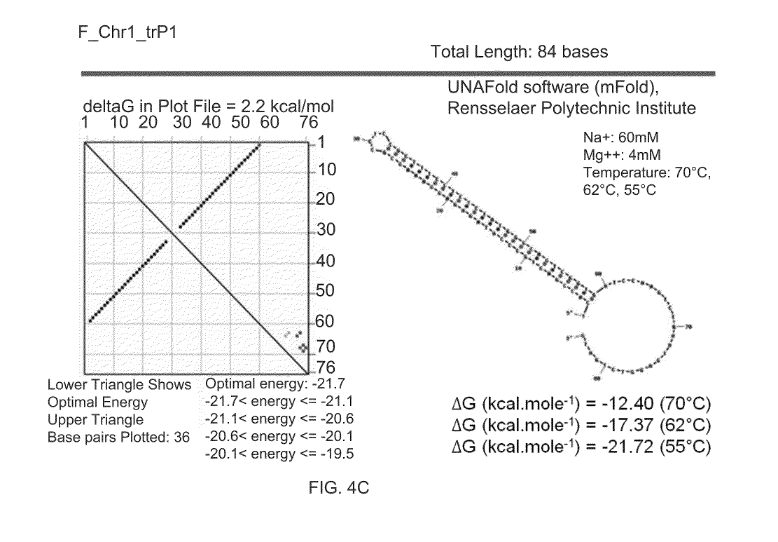

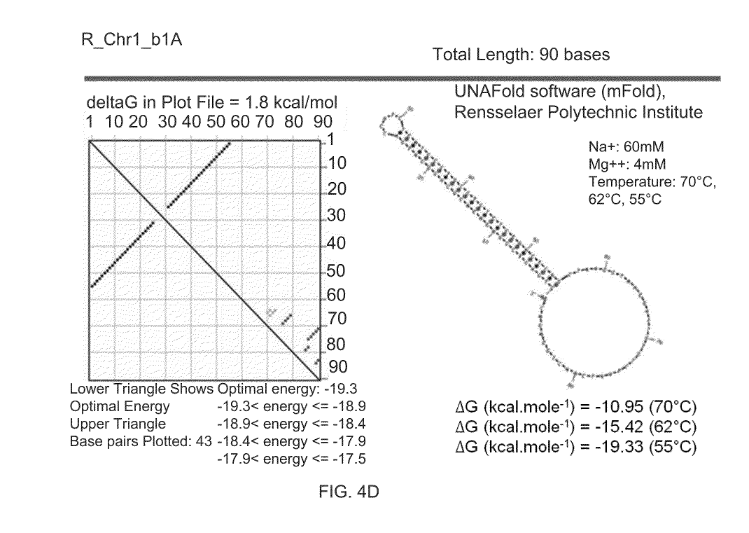

The first self-complementary region 103 and the second self-complementary region 105 have nucleotide sequences that are sufficiently complementary such that they hybridize intramolecularly to form a double-stranded region (e.g., at the appropriate thermodynamic, kinetic, and/or solution and reaction conditions). In particular, in some embodiments the first self-complementary region 103 and the second self-complementary region 105 are completely complementary; in some embodiments, the first self-complementary region 103 and the second self-complementary region 105 are not completely complementary. Under appropriate solution conditions, a double-stranded duplex will form from the first self-complementary region 103 and the second self-complementary region 105 when the first self-complementary region 103 and the second self-complementary region 105 are completely complementary or, alternatively, when the first self-complementary region 103 and the second self-complementary region 105 are not completely complementary but are sufficiently complementary to hybridize (e.g., a duplex forms comprising a number of mismatches). See, e.g., FIG. 2B and FIG. 4.

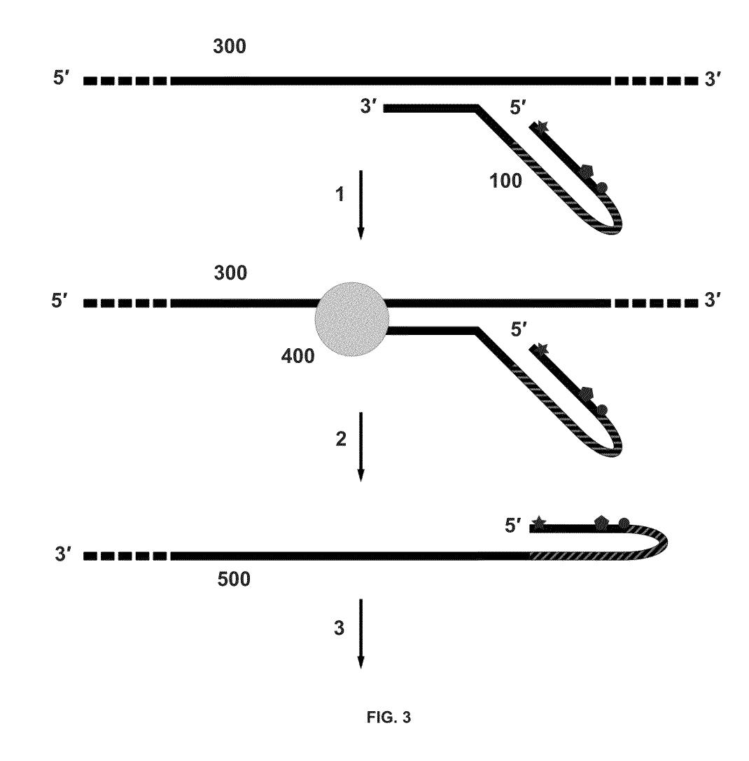

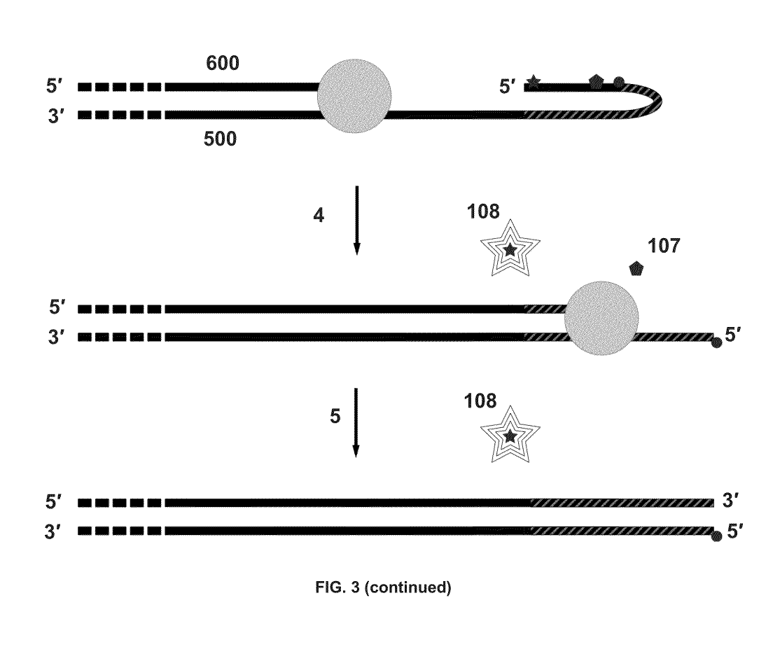

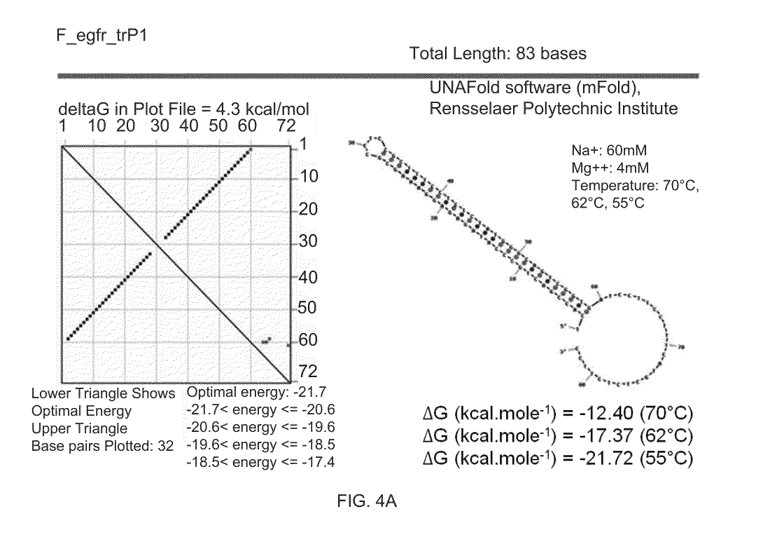

In some embodiments, the hairpin oligonucleotide 100 comprises an amplicon-specific segment 101 comprising a sequence that is complementary to a target to be amplified and/or is complementary to region flanking a target to be amplified. The amplicon-specific segment 101 comprises a sequence that is sufficiently complementary to the target or region flanking the target such that oligonucleotide 100 hybridizes to the template to form a primer-template hybrid comprising a double-stranded region (e.g., at the appropriate thermodynamic, kinetic, and/or solution and reaction conditions). See FIG. 3.

In some embodiments the amplicon-specific segment 101 and the target or region flanking the target are completely complementary; in some embodiments, the amplicon-specific segment 101 and the target or region flanking the target are not completely complementary. Under appropriate solution conditions, a double-stranded duplex will form from the amplicon-specific segment 101 and the target or region flanking the target when the amplicon-specific segment 101 and the target or region flanking the target are completely complementary or, alternatively, when the amplicon-specific segment 101 and the target or region flanking the target are not completely complementary but are sufficiently complementary to hybridize (e.g., a duplex forms comprising a number of mismatches). The primer-template hybrid provides a substrate that is recognized by a polymerase and from which synthesis of a nucleic acid is initiated (e.g., from the 3′ end of the amplicon-specific sequence). In this way, the amplicon-specific segment acts a primer in an amplification reaction. See FIG. 3, e.g., steps 1 and 2.

In some embodiments, the hairpin oligonucleotide 100 or 200 comprises an adaptor sequence (e.g., a NGS platform-specific adaptor sequence) that is appended to the amplicons produced by an amplification reaction in which the oligonucleotide 100 or 200 is used. In some embodiments, the adaptor provides functionality (e.g., a universal sequence) for integrating an amplicon library into a NGS system workflow. In some embodiments, the adaptor also provides functionality (e.g., a tag) for the manipulation, isolation, and/or characterization of the amplicons as a collection.

Amplicons produced from an oligonucleotide comprising an adaptor thus comprise a portion derived from the template (e.g., which may have an unknown sequence) and a portion defined by the user of the technology (e.g., which may have a known sequence). Thus, in some embodiments, the technology produces amplicons comprising different sequences derived from the template (e.g., an amplicon library) and comprising the same adaptor sequence (e.g., comprising a universal sequence) that is recognized by the NGS platform and/or a tag for manipulation, isolation, and/or characterization (e.g., identification (indexing)) of the amplicons. For example, in some embodiments, the adaptors comprise one or more universal sequences and/or one or more tags shared among multiple different adaptors or subsets of different adaptors. That is, regardless of the uniqueness of the amplified target-derived sequence of any one amplicon, the adaptor provides one or more common functionality or functionalities for manipulating, isolating, and/or characterizing (e.g., identifying (e.g., by one or more index or indices)) the amplicon(s), e.g., without necessarily knowing the sequence of the target-derived portion.

Accordingly, in some embodiments the hairpin oligonucleotide 100 or 200 comprises an adaptor comprising a “universal” sequence (e.g., a NGS platform-dependent sequence) that is appended to the amplicons produced by an amplification reaction in which the oligonucleotide 100 or 200 is used (e.g., in some embodiments the adaptor comprises a universal sequence).

In some embodiments, the hairpin oligonucleotide 100 or 200 comprises a “tag” (e.g., in some embodiments, the adaptor comprises a tag). Generally, the tag sequence is not derived from or complementary to the target to be amplified (or is not derived from or complementary to the region flanking a target to be amplified). The tag sequence is typically defined by the user of the technology to add a functional characteristic to amplicons produced by an amplification reaction.

For instance, in some embodiments the tag comprises a restriction enzyme recognition sequence that is appended to the amplicons produced by an amplification reaction in which the oligonucleotide is used. Other examples of tag components (e.g., sequences) that are appended to the amplicons produced by an amplification reaction in which the oligonucleotide is used include a linker, an index, a capture sequence, a primer binding site, an antigen, a poly-A tail, an epitope, a sequence recognized by a capture probe (e.g., a capture probe linked to a solid support) for the isolation and/or purification of amplicons, etc. That is, in some embodiments the tag comprises a linker, an index, a capture sequence, a primer binding site, an antigen, a poly-A tail, an epitope, a sequence recognized by a capture probe (e.g., a capture probe linked to a solid support), etc.

In some embodiments, the tag comprises an index (e.g., a barcode nucleotide sequence).

Accordingly, tags (and thus, adaptors) can contain one or more of a variety of sequence elements, including but not limited to, one or more amplification primer annealing sequences or complements thereof, one or more sequencing primer annealing sequences or complements thereof, one or more index sequences, one or more restriction enzyme recognition sites, one or more overhangs complementary to one or more target polynucleotide overhangs, one or more probe binding sites (e.g. for attachment to a sequencing platform, such as a flow cell for massive parallel sequencing, such as developed by Illumina, Inc.), and combinations thereof. Two or more sequence elements can be non-adjacent to one another (e.g. separated by one or more nucleotides), adjacent to one another, partially overlapping, or completely overlapping. For example, an amplification primer annealing sequence can also serve as a sequencing primer annealing sequence. Sequence elements can be located at or near the 3′ end, at or near the 5′ end, or in the interior of the tag or adaptor.

In some embodiments, the first tag sequences in a plurality of tag sequences having different index sequences comprise a sequence element common among all first tag sequences in the plurality. In some embodiments, the second tag sequences comprise a sequence element common among all second tag sequences that is different from the common sequence element shared by the first tag sequences. A difference in sequence elements can be any such that at least a portion of the different tag sequences do not completely align, for example, due to changes in sequence length, deletion, or insertion of one or more nucleotides, or a change in the nucleotide composition at one or more nucleotide positions (such as a base change or base modification).

In some embodiments, the tags comprise a molecular binding site identification element to facilitate identification and/or isolation of the target nucleic acid (e.g., one or more amplicons) for downstream applications. Molecular binding as an affinity mechanism allows for the interaction between two molecules to result in a stable association complex. Molecules that can participate in molecular binding reactions include proteins, nucleic acids, carbohydrates, lipids, and small organic molecules such as ligands, peptides, or drugs.

When a nucleic acid molecular binding site is used as part of the tag segment, it can be used to employ selective hybridization to isolate a target sequence (e.g., one or more amplicons). Selective hybridization may restrict substantial hybridization to target nucleic acids containing the tag sequence with the molecular binding site and capture nucleic acids that are sufficiently complementary to the molecular binding site. Thus, through “selective hybridization” one can detect the presence of the target polynucleotide in a sample containing a pool of many nucleic acids. An example of a selective hybridization isolation system comprises a system with one or more capture oligonucleotides (e.g., a “capture probe”) that comprise complementary sequences to the molecular binding identification elements and are optionally immobilized to a solid support. In other embodiments, the capture oligonucleotides are complementary to the target sequence itself or an index or other unique sequence contained within the tag. The capture oligonucleotides can be immobilized to various solid supports, such as inside of a well of a plate, mono-dispersed spheres or beads (e.g., magnetic (e.g., paramagnetic) beads), microarrays, or any other suitable support surface known in the art. The hybridized nucleic acids attached on the solid support can be isolated by washing away the undesirable non-binding nucleic acids, leaving the desirable target nucleic acids. If complementary capture oligonucleotides molecules are fixed to paramagnetic spheres or similar bead technology for isolation, then spheres can then be mixed in a tube together with the target nucleic acid comprising the tag sequence. When the tag sequences have been hybridized with the complementary sequences fixed to the spheres, undesirable molecules can be washed away while spheres are kept in the tube with a magnet or similar agent. The desired target nucleic acids can be subsequently released by increasing the temperature, changing the pH, or by using any other suitable elution method known in the art.

In the exemplary embodiment depicted in FIG. 1A, the hairpin oligonucleotide 100 comprises an adaptor sequence in segment 103 and/or segment 104. In the exemplary embodiment depicted in FIG. 1B, the hairpin oligonucleotide 200 comprises an adaptor sequence in segment 203 and/or segment 204. In some embodiments, the adaptor may also include a tag region 102 or 202.

In some embodiments, the stem-loop structure of the hairpin oligonucleotide 100 or 200 occludes the universal sequence of the adaptor from inter-molecular hybridization. For example, in some embodiments the stem-loop structure of the hairpin oligonucleotide 100 or 200 occludes the universal sequence from inter-molecular hybridization with free (e.g., non-incorporated) hairpin oligonucleotides in the reaction and/or occludes the universal sequence from inter-molecular hybridization with amplification products comprising the universal sequence.

In the embodiment depicted in FIG. 1A as the hairpin oligonucleotide 100, the fluorescent moiety 108 and the quencher moiety 107 are chosen and positioned in the oligonucleotide such that the quencher moiety quenches the fluorescence of the fluorescent moiety 108 when the hairpin oligonucleotide comprises the fluorescent moiety 108 and the quencher moiety 107. In some embodiments, the fluorescent moiety 108 and the quencher moiety 107 are linked (e.g., appended, attached, joined, etc.) to nucleotides of the oligonucleotide.

In another embodiment, the technology provides hairpin (e.g., “stem-loop”) oligonucleotides that do not comprise fluorescence and quencher moieties (see, e.g., FIG. 1B). The hairpin oligonucleotide 200 comprises a single-stranded region (e.g., black segments 201 and 202), a double-stranded duplex region (e.g., white hatched segment 203 hybridized to complementary white filled segment 205), and a single-stranded loop region (e.g., black hatched segment 204). Additionally, in some embodiments, the oligonucleotide 200 comprises several segments, including a first portion comprising, derived from, and/or complementary to the target template (e.g., an amplicon-specific priming segment) 201, a tag 202, a first self-complementary region 203, a single-stranded loop region 204, a second self-complementary region 205, and a blocker (e.g., nuclease-resistant (e.g., exonuclease-resistant (e.g., 5′ to 3′ exonuclease-resistant))) moiety 206.

Accordingly, in some embodiments the hairpin oligonucleotide 200 comprises a first portion 201 comprising, derived from, and/or complementary to the target template (e.g., an amplicon-specific priming segment); and a second portion comprising a user-defined adaptor (e.g., an adaptor comprising a tag 202 (e.g., a tag comprising a linker, index, capture sequence, restriction site, primer binding site, antigen, and/or other functional site) and/or comprising a universal sequence (e.g., comprising a platform-dependent sequence)).

The first self-complementary region 203 and the second self-complementary region 205 have nucleotide sequences that are sufficiently complementary such that they hybridize intramolecularly to form a double-stranded region (e.g., at the appropriate thermodynamic, kinetic, and/or reaction conditions). In particular, in some embodiments the first self-complementary region 203 and the second self-complementary region 205 are completely complementary; in some embodiments, the first self-complementary region 203 and the second self-complementary region 205 are not completely complementary. Under appropriate solution conditions, a double-stranded duplex will form from the first self-complementary region 203 and the second self-complementary region 205 when the first self-complementary region 203 and the second self-complementary region 205 are completely complementary or, alternatively, when the first self-complementary region 203 and the second self-complementary region 205 are not completely complementary but are sufficiently complementary to hybridize (e.g., a duplex forms comprising a number of mismatches).

The hairpin oligonucleotides are designed to assume several states in response to thermodynamic variables (e.g., temperature, pressure, volume), kinetic parameters (e.g., binding (e.g., on and off) rates), and solution conditions (e.g., salt concentration, water activity, pH, other solution components, etc.). See, e.g., FIG. 2 and FIG. 4. Under some conditions (e.g., at a denaturing or melting temperature in a standard PCR reaction mixture, e.g., at approximately 94° C. to 95° C. or above), the hairpin oligonucleotides assume a linear conformation (see, e.g., FIG. 2A). In this conformation, the first self-complementary region 103 and the second self-complementary region 105 are not hybridized and the oligonucleotide does not comprise a double-stranded duplex comprising the first self-complementary region 103 and the second self-complementary region 105.

Under different conditions, e.g., at a lower temperature (e.g., at a PCR extension temperature, e.g., at a temperature that is approximately 68° C. to 70° C. to 75° C.), intramolecular kinetic rate factors and thermodynamic stability favor the formation of the hairpin structure (see, e.g., FIG. 2B). In this hairpin conformation the first self-complementary region 103 and the second self-complementary region 105 are hybridized to form a double-stranded duplex comprising the first self-complementary region 103 and the second self-complementary region 105. The universal sequence of the adaptor is “hidden” from hybridizing with complementary sequences in the reaction mixture. The amplicon-specific segment 101 and the tag 102 (if present) are single-stranded.

Then, under further different conditions, e.g., at a still lower temperature (e.g., at a temperature that is a PCR primer binding temperature, e.g., at approximately 55° C. to 65° C.), kinetic rate factors and thermodynamic stability favor the hybridization of the amplicon-specific segment 101 to its complementary target sequence on the template 300. In this conformation, the oligonucleotide comprises the double-stranded duplex structure comprising the first self-complementary region 103 and the second self-complementary region 105 and the amplicon-specific segment 101 provides a 3′ end (e.g., a 3′ OH) from which a polymerase synthesizes a strand complementary to the template nucleic acid 300. The hairpin oligonucleotide depicted in FIG. 1B as hairpin oligonucleotide 200 is designed similarly as the hairpin oligonucleotide 100 to assume these states in response to thermodynamic and kinetic parameters such as temperature, solution components, and binding rates (see, e.g., FIG. 2). Accordingly, the interactions and characteristics of the 201, 202, 203, 204, and 205 segments of the hairpin oligonucleotide 200 behave in a similar manner as the 101, 102, 103, 104, and 105 segments of the hairpin oligonucleotide 100. Embodiments of the hairpin oligonucleotides shown in FIGS. 1C to 1F include similar features and are designed to behave similarly to embodiments 100 and 200 shown in FIG. 1A and FIG. 1B.

The oligonucleotides are designed so that the intra-molecular hybridization event (e.g., formation of the double-stranded duplex; see FIG. 2B) occurs prior to the inter-molecular hybridization event (e.g., hybridization of the single stranded portion of the oligonucleotide to its complementary sequence to form a primer-template hybrid; see FIG. 2C) as the temperature is lowered. For example, in some embodiments, the stem portion of the hairpin (e.g., the duplex region) is designed to have a higher melting temperature (Tm) than the single-stranded portion when hybridized to its complement. Design parameters that affect the intra-molecular Tₘ (for the duplex structure) and the inter-molecular Tₘ (for the amplicon-specific segment hybridized to its target) include, e.g: the length of the duplex region; the length of the primer-template hybrid (generally longer sequences have a higher Tₘ when GC contents are similar); the number of base pairs and/or the number of mismatches within the duplex region; the number of base pairs and/or the number of mismatches within the primer-template hybrid; and/or the number of modifications (e.g., in the nucleotide, base, or linkage between nucleotides) incorporated into the oligonucleotide within the portions that form the duplex and/or primer-template hybrid.

These design parameters provide control over the behavior of the oligonucleotide, e.g., providing an oligonucleotide that first forms the hairpin duplex and subsequently forms the primer-template hybrid during a typical PCR temperature profile (see, e.g., FIGS. 2A. 2B, and 2C).

### Fluorescent Moieties

In some embodiments, the hairpin primers comprise a fluorescent moiety (e.g., a fluorogenic dye, also referred to as a “fluorophore” or a “fluor”). A wide variety of fluorescent moieties is known in the art and methods are known for linking a fluorescent moiety to a nucleotide prior to incorporation of the nucleotide into an oligonucleotide and for adding a fluorescent moiety to an oligonucleotide after synthesis of the oligonucleotide.

Examples of compounds that may be used as the fluorescent moiety include but are not limited to xanthene, anthracene, cyanine, porphyrin, and coumarin dyes. Examples of xanthene dyes that find use with the present technology include but are not limited to fluorescein, 6-carboxyfluorescein (6-FAM), 5-carboxyfluorescein (5-FAM), 5- or 6-carboxy-4, 7, 2′,7′-tetrachlorofluorescein (TET), 5- or 6-carboxy-4′5′2′4′5′7′ hexachlorofluorescein (HEX), 5′ or 6′-carboxy-4′,5′-dichloro-2,′7′-dimethoxyfluorescein (JOE), 5-carboxy-2′,4′,5′,7′-tetrachlorofluorescein (ZOE), rhodol, rhodamine, tetramethylrhodamine (TAMRA), 4,7-dlchlorotetramethyl rhodamine (DTAMRA), rhodamine X (ROX), and Texas Red. Examples of cyanine dyes that may find use with the present invention include but are not limited to Cy 3, Cy 3.5, Cy 5, Cy 5.5, Cy 7, and Cy 7.5. Other fluorescent moieties and/or dyes that find use with the present technology include but are not limited to energy transfer dyes, composite dyes, and other aromatic compounds that give fluorescent signals. In some embodiments, the fluorescent moiety comprises a quantum dot.

As such, according to the technology, exemplary fluorophores and dyes that find use include, without limitation, fluorescent dyes and/or molecules that quench the fluorescence of the fluorescent dyes. Fluorescent dyes include, without limitation, d-Rhodamine acceptor dyes including Cy5, dichloro[R110], dichloro[R6G], dichloro[TAMRA], dichlorol[ROX] or the like, fluorescein donor dyes including fluorescein, 6-FAM, 5-FAM, or the like; Acridine including Acridine orange, Acridine yellow, Proflavin, pH 7, or the like; Aromatic Hydrocarbons including 2-Methylbenzoxazole, Ethyl p-dimethylaminobenzoate, Phenol, Pyrrole, benzene, toluene, or the like; Arylmethine Dyes including Auramine O, Crystal violet, Crystal violet, glycerol, Malachite Green or the like; Coumarin dyes including 7-Methoxycoumarin-4-acetic acid, Coumarin 1, Coumarin 30, Coumarin 314, Coumarin 343, Coumarin 6 or the like; Cyanine Dyes including 1,1′-diethyl-2,2′-cyanine iodide, Cryptocyanine, Indocarbocyanine (C3) dye, Indodicarbocyanine (C5) dye, Indotricarbocyanine (C7) dye, Oxacarbocyanine (C3) dye, Oxadicarbocyanine (C5) dye, Oxatricarbocyanine (C7) dye, Pinacyanol iodide, Stains all, Thiacarbocyanine (C3) dye, ethanol, Thiacarbocyanine (C3) dye, n-propanol, Thiadicarbocyanine (C5) dye, Thiatricarbocyanine (C7) dye, or the like; Dipyrrin dyes including N,N′-Difluoroboryl-1,9-dimethyl-5-(4-iodophenyl)-dipyrrin, N,N′-Difluoroboryl-1,9-dimethyl-5-1(4-(2-trimethylsilylethynyl), N,N′-Difluoroboryl-1,9-dimethyl-5-phenydipyrrin, or the like; Merocyanines including 4-(dicyanomethylene)-2-methyl-6-(p-dimethylaminostyryl)-4H-pyran (DCM), acetonitrile, 4-(dicyanomethylene)-2-methyl-6-(p-dimethylaminostyryl)-4H-pyran (DCM), methanol, 4-Dimethylamino-4′-nitrostilbene, Merocyanine 540, or the like; Miscellaneous Dyes including 4′,6-Diamidino-2-phenylindole (DAPI), dimethylsulfoxide, 7-Benzylamino-4-nitrobenz-2-oxa-1,3-diazole, Dansyl glycine, Dansyl glycine, dioxane, Hoechst 33258, DMF, Hoechst 33258, Lucifer yellow CH, Piroxicam, Quinine sulfate, Quinine sulfate, Squarylium dye III, or the like; Oligophenylenes including 2,5-Diphenyloxazole (PPO), Biphenyl, POPOP, p-Quaterphenyl, p-Terphenyl, or the like; Oxazines including Cresyl violet perchlorate, Nile Blue, methanol, Nile Red, ethanol, Oxazine 1, Oxazine 170, or the like; Polycyclic Aromatic Hydrocarbons including 9,10-Bis(phenylethynyl)anthracene, 9,10-Diphenylanthracene, Anthracene, Naphthalene, Perylene, Pyrene, or the like; polyene/polyynes including 1,2-diphenylacetylene, 1,4-diphenylbutadiene, 1,4-diphenylbutadiyne, 1,6-Diphenylhexatriene, Beta-carotene, Stilbene, or the like; Redox-active Chromophores including Anthraquinone, Azobenzene, Benzoquinone, Ferrocene, Riboflavin, Tris(2,2′-bipyridypruthenium(II), Tetrapyrrole, Bilirubin, Chlorophyll a, diethyl ether, Chlorophyll a, methanol, Chlorophyll b, Diprotonated-tetraphenylporphyrin, Hematin, Magnesium octaethylporphyrin, Magnesium octaethylporphyrin (MgOEP), Magnesium phthalocyanine (MgPc), PrOH, Magnesium phthalocyanine (MgPc), pyridine, Magnesium tetramesitylporphyrin (MgTMP), Magnesium tetraphenylporphyrin (MgTPP), Octaethylporphyrin, Phthalocyanine (Pc), Porphin, ROX, TAMRA, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), Tetraphenylporphyrin (TPP), Vitamin B12, Zinc octaethylporphyrin (ZnOEP), Zinc phthalocyanine (ZnPc), pyridine, Zinc tetramesitylporphyrin (ZnTMP), Zinc tetramesitylporphyrin radical cation, Zinc tetraphenylporphyrin (ZnTPP), or the like; Xanthenes including Eosin Y, Fluorescein, basic ethanol, Fluorescein, ethanol, Rhodamine 123, Rhodamine 6G, Rhodamine B, Rose bengal, Sulforhodamine 101, or the like; or mixtures or combination thereof or synthetic derivatives thereof.

Several classes of fluorogenic dyes and specific compounds are known that are appropriate for particular embodiments of the technology: xanthene derivatives such as fluorescein, rhodamine, Oregon green, eosin, and Texas red; cyanine derivatives such as cyanine, indocarbocyanine, oxacarbocyanine, thiacarbocyanine, and merocyanine; naphthalene derivatives (dansyl and prodan derivatives); coumarin derivatives; oxadiazole derivatives such as pyridyloxazole, nitrobenzoxadiazole, and benzoxadiazole; pyrene derivatives such as cascade blue; oxazine derivatives such as Nile red, Nile blue, cresyl violet, and oxazine 170; acridine derivatives such as proflavin, acridine orange, and acridine yellow; arylmethine derivatives such as auramine, crystal violet, and malachite green; and tetrapyrrole derivatives such as porphin, phtalocyanine, bilirubin. In some embodiments the fluorescent moiety a dye that is xanthene, fluorescein, rhodamine, BODIPY, cyanine, coumarin, pyrene, phthalocyanine, phycobiliprotein, ALEXA FLUOR® 350, ALEXA FLUOR® 405, ALEXA FLUOR® 430, ALEXA FLUOR® 488, ALEXA FLUOR® 514, ALEXA FLUOR® 532, ALEXA FLUOR® 546, ALEXA FLUOR® 555, ALEXA FLUOR® 568, ALEXA FLUOR® 568, ALEXA FLUOR® 594, ALEXA FLUOR® 610, ALEXA FLUOR® 633, ALEXA FLUOR® 647, ALEXA FLUOR® 660, ALEXA FLUOR® 680, ALEXA FLUOR® 700, ALEXA FLUOR® 750, or a squaraine dye. In some embodiments, the label is a fluorescently detectable moiety as described in, e.g., Haugland (September 2005) MOLECULAR PROBES HANDBOOK OF FLUORESCENT PROBES AND RESEARCH CHEMICALS (10th ed.), which is herein incorporated by reference in its entirety.

In some embodiments the label (e.g., a fluorescently detectable label) is one available from ATTO-TEC GmbH (Am Eichenhang 50, 57076 Siegen, Germany), e.g., as described in U.S. Pat. Appl. Pub. Nos. 20110223677, 20110190486, 20110172420, 20060179585, and 20030003486; and in U.S. Pat. No. 7,935,822, all of which are incorporated herein by reference.

One of ordinary skill in the art will recognize that dyes having emission maxima outside these ranges may be used as well. In some cases, dyes ranging between 500 nm to 700 nm have the advantage of being in the visible spectrum and can be detected using existing photomultiplier tubes. In some embodiments, the broad range of available dyes allows selection of dye sets that have emission wavelengths that are spread across the detection range. Detection systems capable of distinguishing many dyes are known in the art.

### Quencher Moieties

In some embodiments, the hairpin primers comprise a quencher moiety. A wide variety of quencher moieties is known in the art. For example, in some embodiments an oligonucleotide comprises a quencher than is a Black Hole Quencher (e.g., BHQ-0, BHQ-1, BHQ-2, BHQ-3), a Dabcyl, an Iowa Black Quencher (e.g., Iowa Black FQ, Iowa Black RQ), an Eclipse quencher.

In some embodiments a BHQ-1 is used with a fluorescent moiety that has an emission wavelength from approximately 500-600 nm. In some embodiments a BHQ-2 is used with a fluorescent moiety that has an emission wavelength from approximately 550-675 nm. In some embodiments, a FRET pair is a fluorophore-quencher pair that provides quenching.

Some exemplary fluorophore-quencher pairs include FAM and BHQ-1, TET and BHQ-1, JOE and BHQ-1, HEX and BHQ-1, Cy3 and BHQ-2, TAMRA and BHQ-2, ROX and BHQ-2, Cy5 and BHQ-3, Cy5.5 and BHQ-3, FAM and BHQ-1, TET and BHQ-1, JOE and 3′-BHQ-1, HEX and BHQ-1, Cy3 and BHQ-2, TAMRA and BHQ-2, ROX and BHQ-2, Cy5 and BHQ-3, Cy5.5 and BHQ-3, or similar fluorophore-quencher pairs available from the commercial entities such as Biosearch Technologies, Inc. of Novato, Calif.

### Blocker Moieties

The hairpin oligonucleotide comprises naturally occurring dNMP (e.g., dAMP, dGMP, dCMP and dTMP), modified nucleotides, and/or non-natural nucleotides. In some embodiments, the hairpin oligonucleotides comprise a blocker (e.g., nuclease-resistant) moiety that is resistant to degradation, e.g., by an enzyme (e.g., an enzyme having exonuclease activity (e.g., an exonuclease enzyme or a polymerase enzyme comprising an exonuclease activity)). In some embodiments, the blocker moiety comprises a modified nucleotide and/or a non-natural nucleotide. In some embodiments, the blocker moiety comprises a modified phosphodiester link between nucleotides and/or a non-natural phosphodiester link between nucleotides. In some embodiments, the hairpin oligonucleotide comprises ribonucleotides.

Further, in some embodiments the hairpin oligonucleotide used in this technology may include nucleotides with backbone modifications such as to provide peptide nucleic acid (PNA) (Egholm et al. (1993) Nature, 365: 566-568), phosphorothioate DNA, phosphorodithioate DNA, phosphoramidate DNA, amide-linked DNA, MMI-linked DNA, 2′-O-methyl RNA, alpha-DNA, and methyl phosphonate DNA, nucleotides with sugar modifications such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides having base modifications such as C-5 substituted pyrimidines (substituents including fluoro-, bromo-, chloro-, iodo-, methyl-, ethyl-, vinyl-, formyl-, ethynyl-, propynyl-, alkynyl-, thiazolyl-, imidazolyl-, and pyridyl-), 7-deazapurines with C-7 substituents (substituents including fluoro-, bromo-, chloro-, iodo-, methyl-, ethyl-, vinyl-, formyl-, alkynyl-, alkenyl-, thiazolyl-, imidazolyl-, and pyridyl-), inosine, and diaminopurine.

### PEG Linkers

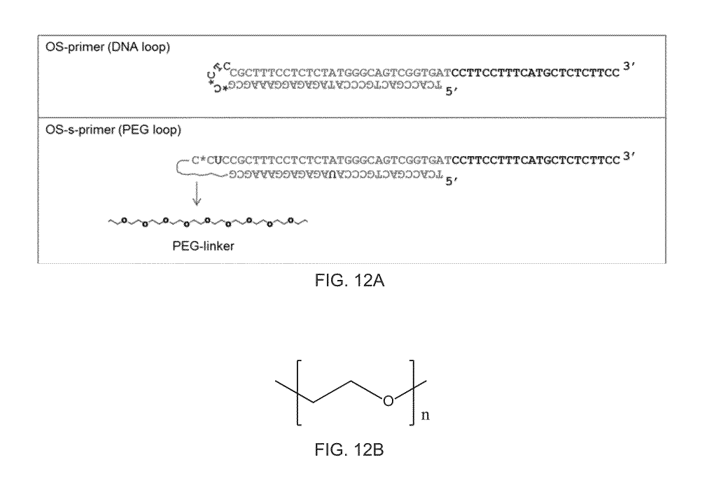

In some embodiments the hairpin oligonucleotides comprise a polyethylene glycol (PEG) linker. See, e.g., FIG. 1E and FIG. 1F. In some embodiments, an oligonucleotide comprising a PEG linker is useful for an amplification reaction (e.g., as described herein) using a polymerase (e.g., a high-fidelity polymerase) that comprises a proof-reading activity, a 3′ exonuclease activity, and/or a strand displacement activity, but that lacks a 5′ exonuclease activity. In these designs, the loop portion (e.g., 224 or 234) of the hairpin oligonucleotide comprises a PEG linker instead of a single stranded (nucleic acid) segment comprising linked nucleotides (FIG. 1E, FIG. 1F, FIG. 12). Thus, in some embodiments, the DNA-PEG junction stops polymerase extension.

Polyethylene glycol is also known as polyethylene oxide (PEO) or polyoxyethylene (POE). PEG is a polymer having a structure H— (O— CH₂—CH₂)ₙ—OH, wherein the unit in parentheses is repeated (e.g., is repeated n times, e.g., wherein n equals 1 to 40, e.g., n equals 1, 2, 3, 4, 5, 6, 7, 8, 9, 10, 11, 12, 13, 14, 15 16, 17, 18, 19, 20, 21, 22, 23, 24, 25, 26, 27, 28, 29, 30, 31, 32, 33, 34, 35, 36, 37, 38, 39, or 40). When incorporated into embodiments of the hairpin oligonucleotides described herein, the PEG linker has a structure according to FIG. 12B. In some embodiments, the n in FIG. 12B equals 1, 2, 3, 4, 5, 6, 7, 8, 9, 10, 11, 12, 13, 14, 15 16, 17, 18, 19, 20, 21, 22, 23, 24, 25, 26, 27, 28, 29, 30, 31, 32, 33, 34, 35, 36, 37, 38, 39, or 40.

### Amplification Reactions

In some embodiments, the technology relates to reaction mixtures comprising a hairpin oligonucleotide described herein (e.g., a hairpin oligonucleotide 100 or 200). For example, some embodiments relate to an amplification reaction mixture comprising one or more hairpin oligonucleotides described herein (e.g., a hairpin oligonucleotide 100 or 200), a polymerase, nucleotide monomers (e.g., dNTPs), and a template. In some embodiments, the technology relates to reaction mixtures further comprising a typical amplification primer.

In an exemplary embodiment depicted in FIG. 3, a hairpin oligonucleotide 100 is used to amplify a region of a target nucleic acid 300. The hairpin primer 100 is hybridized to its complementary sequence on the target template 300 to form a primer-template hybrid having a free 3′ end (e.g., a 3′ OH substrate for extension of a nucleic acid). The hairpin primer 100 is in the hairpin (stem-loop state) and comprises the fluorescent moiety (star) in a quenched state. In particular, the fluorescent moiety (star) and the quencher moiety (pentagon) are located in space such that the quencher moiety minimizes or eliminates the detection of fluorescence from the fluorescent moiety.

In some embodiments, a reaction mixture comprises a polymerase (e.g., a polymerase comprising 5′ to 3′ exonuclease activity). As shown in FIG. 3, the polymerase 400 binds to the primer-template hybrid (Step 1) and extends the 3′ end of the hairpin primer (e.g., from the amplicon-specific priming region) by nucleic acid synthesis to form nucleic acid 500 comprising a hairpin structure at its 5′ end and the fluorescent moiety in a quenched state (Step 2). Denaturation (e.g., “melting”) of the hybridized duplex comprising template strand 300 and nucleic acid 500 results in the separation of the template strand 300 from the nucleic acid 500 in the reaction mixture. The nucleic acid 500 comprises a single stranded region and a hairpin structure at its 5′ end.

Next, in some embodiments, a primer (e.g., a typical amplification primer, a hairpin oligonucleotide, or other primer providing a substrate for extension by a polymerase) binds to the single stranded portion of nucleic acid 500 and thus provides a substrate for polymerization and synthesis of a nucleic acid 600 complementary to nucleic acid 500 (Step 3).

In some embodiments, the polymerase encounters the 5′ end of the double-stranded (e.g., hairpin) region of the nucleic acid 500 during synthesis of nucleic acid 600. The 5′ end of the hairpin structure provides a substrate for the 5′ to 3′ exonuclease activity of the polymerase. Accordingly, the polymerase degrades the double stranded hairpin structure from the 5′ end of the hairpin, releasing the fluorescent moiety 108 (star) and the quenching moiety 107 (pentagon) (Step 4). Separation in space of the free fluorescent moiety 108 and the free quenching moiety 107 (e.g., as the fluorescent moiety and the quenching moiety diffuse away from one another in the reaction mixture) allows the fluorescent moiety 108 to fluoresce (see FIG. 3, multiply outlined (e.g., “shining”) star). The signal detected from the fluorescent moiety is related to the amount of amplicon produced by the reaction, thus providing a qualitative indicator of successful amplification and/or a quantitative measure of amplicon concentration or amount (e.g., providing a real-time quantitative amplification method).

Degradation of the duplex region by the exonuclease of the polymerase is blocked by the blocker (exonuclease-resistant) moiety (circle) at a defined location, thus leaving a defined end for the nucleic acid. Further, degradation of the duplex region by the exonuclease exposes the adaptor (e.g., comprising a universal (e.g., NGS platform-dependent) segment) (black filled region with hatching) and, optionally, a tag (when present) (black filled region with hatching) and the polymerase continues synthesis to the end of the template, which is delimited by the blocker (e.g., nuclease resistant) moiety (Step 5). The resulting amplicon comprises a segment from the target (e.g., comprising target sequence) (black filled segment) and the adaptor (e.g., comprising a universal (e.g., NGS platform-dependent) segment) (black filled region with hatching) and, optionally, a tag (when present) (black filled region with hatching) After multiple amplification cycles, a major population of linear double-stranded amplicon products is generated, wherein each amplicon comprises an adaptor at one or both ends.

In some embodiments related to hairpin oligonucleotides comprising a PEG linker, the polymerase (e.g., a high-fidelity polymerase) comprises a proof-reading activity, a 3′ exonuclease activity, and/or a strand displacement activity, but lacks a 5′ exonuclease activity, and the PEG-DNA junction blocks the polymerase to provide a defined end to amplicons.

### Samples

In some embodiments, nucleic acids (e.g., DNA or RNA) are isolated from a biological sample containing a variety of other components, such as proteins, lipids, and non-template nucleic acids. Nucleic acid template molecules can be obtained from any material (e.g., cellular material (live or dead), extracellular material, viral material, environmental samples (e.g., metagenomic samples), synthetic material (e.g., amplicons such as provided by PCR or other amplification technologies)), obtained from an animal, plant, bacterium, archaeon, fungus, or any other organism. Biological samples for use in the present technology include viral particles or preparations thereof. Nucleic acid molecules can be obtained directly from an organism or from a biological sample obtained from an organism, e.g., from blood, urine, cerebrospinal fluid, seminal fluid, saliva, sputum, stool, hair, sweat, tears, skin, and tissue. Exemplary samples include, but are not limited to, whole blood, lymphatic fluid, serum, plasma, buccal cells, sweat, tears, saliva, sputum, hair, skin, biopsy, cerebrospinal fluid (CSF), amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs, aspirates (e.g., bone marrow, fine needle, etc.), washes (e.g., oral, nasopharyngeal, bronchial, bronchialalveolar, optic, rectal, intestinal, vaginal, epidermal, etc.), and/or other specimens.

Any tissue or body fluid specimen may be used as a source for nucleic acid for use in the technology, including forensic specimens, archived specimens, preserved specimens, and/or specimens stored for long periods of time, e.g., fresh-frozen, methanol/acetic acid fixed, or formalin-fixed paraffin embedded (FFPE) specimens and samples. Nucleic acid template molecules can also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA. A sample may also be isolated DNA from a non-cellular origin, e.g. amplified/isolated DNA that has been stored in a freezer.

Nucleic acid molecules can be obtained, e.g., by extraction from a biological sample, e.g., by a variety of techniques such as those described by Maniatis, et al. (1982) Molecular Cloning: A Laboratory Manual, Cold Spring Harbor, N.Y. (see, e.g., pp. 280-281).

In some embodiments, the technology provides for the size selection of nucleic acids, e.g., to remove very short fragments or very long fragments. In various embodiments, the size is limited to be 0.5, 1, 2, 3, 4, 5, 7, 10, 12, 15, 20, 25, 30, 50, 100 kb or longer.

In various embodiments, a nucleic acid is amplified. Any amplification method known in the art may be used. Examples of amplification techniques that can be used include, but are not limited to, PCR, quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), hot start PCR, nested PCR, in situ polony PCR, in situ rolling circle amplification (RCA), bridge PCR, picotiter PCR, and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR), and nucleic acid based sequence amplification (NABSA). Other amplification methods that can be used herein include those described in U.S. Pat. Nos. 5,242,794; 5,494,810; 4,988,617; and 6,582,938.

In some embodiments, the technology finds use in preparing an amplicon panel, e.g., an amplicon panel library, for sequencing. An amplicon panel is a collection of amplicons that are related, e.g., to a disease (e.g., a polygenic disease), disease progression, developmental defect, constitutional disease (e.g., a state having an etiology that depends on genetic factors, e.g., a heritable (non-neoplastic) abnormality or disease), metabolic pathway, pharmacogenomic characterization, trait, organism (e.g., for species identification), group of organisms, geographic location, organ, tissue, sample, environment (e.g., for metagenomic and/or ribosomal RNA (e.g., ribosomal small subunit (SSU), ribosomal large subunit (LSU), 5S, 16S, 18S, 23S, 28S, internal transcribed sequence (ITS) rRNA) studies), gene, chromosome, etc. For example, a cancer panel comprises specific genes or mutations in genes that have established relevancy to a particular cancer phenotype (e.g., one or more of ABL1, AKT1, AKT2, ATM, PDGFRA, EGFR, FGFR (e.g., FGFR1, FGFR2, FGFR3), BRAF (e.g., comprising a mutation at V600, e.g., a V600E mutation), RUNX1, TET2, CBL, EGFR, FLT3, JAK2, JAK3, KIT, RAS (e.g., KRAS (e.g., comprising a mutation at G12, G13, or A146, e.g., a G12A, G12S, G12C, G12D, G13D, or A146T mutation), HRAS (e.g., comprising a mutation at G12, e.g., a G12V mutation), NRAS (e.g., comprising a mutation at Q61, e.g., a Q61R or Q61K mutation)), MET, PIK3CA (e.g., comprising a mutation at H1047, e.g., a H1047L, H1047L, or H1047R mutation), PTEN, TP53 (e.g., comprising a mutation at R248, Y126, G245, or A159, e.g., a R248W, G245S, or A159D mutation), VEGFA, BRCA, RET, PTPN11, HNHF1A, RB1, CDH1, ERBB2, ERBB4, SMAD4, SKT11 (e.g., comprising a mutation at Q37), ALK, IDH1, IDH2, SRC, GNAS, SMARCB1, VHL, MLH1, CTNNB1, KDR, FBXW7, APC, CSF1R, NPM1, MPL, SMO, CDKN2A, NOTCH1, CDK4, CEBPA, CREBBP, DNMT3A, FES, FOXL2, GATA1, GNAll, GNAQ, HIF1A, IKBKB, MEN1, NF2, PAX5, PIK3R1, PTCH1, STK11, etc.). Some amplicon panels are directed toward particular “cancer hotspots”, that is, regions of the genome containing known mutations that correlate with cancer progression and therapeutic resistance.

In some embodiments, an amplicon panel for a single gene includes amplicons for the exons of the gene (e.g., 1, 2, 3, 4, 5, 6, 7, 8, 9, 10, 11, 12, 13, 14, 15, 16, 17, 18, 19, 20, or more exons). In some embodiments, an amplicon panel for species (or strain, sub-species, type, sub-type, genus, or other taxonomic level) identification may include amplicons corresponding to a suite of genes or loci that collectively provide a specific identification of one or more species (or strain, sub-species, type, sub-type, genus, or other taxonomic level) relative to other species (or strain, sub-species, type, sub-type, genus, or other taxonomic level) (e.g., for bacteria (e.g., MRSA), viruses (e.g., HIV, HCV, HBV, respiratory viruses, etc.)) or that are used to determine drug resistance(s) and/or sensitivity/ies (e.g., for bacteria (e.g., MRSA), viruses (e.g., HIV, HCV, HBV, respiratory viruses, etc.)).

The amplicons of the panel typically comprise 100 to 1000 base pairs, e.g., in some embodiments the amplicons of the panel comprise approximately 100, 125, 150, 175, 200, 225, 250, 275, 300, 325, 350, 375, 400, 325, 350, 375, 400, 425, 450, 475, 500, 525, 550, 575, 600, 625, 650, 675, 700, 725, 750, 775, 800, 825, 850, 875, 900, 925, 950, 975, or 1000 base pairs. In some embodiments, an amplicon panel comprises a collection of amplicons that span a genome, e.g., to provide a genome sequence.

The amplicon panel is often produced through use of amplification oligonucleotides (e.g., such as the hairpin oligonucleotides provided herein), e.g., to produce the amplicon panel from the sample, for sequencing disease-related genes, e.g., to assess the presence of particular mutations and/or alleles in the genome. In some embodiments, 10, 20, 30, 40, 50, 60, 70, 80, 90, 100, 150, 200, 300, 400, 500, 1000, or more genes, loci, regions, etc. are targeted to produce, e.g., 10, 20, 30, 40, 50, 60, 70, 80, 90, 100, 150, 200, 300, 400, 500, 1000, or more amplicons. In some embodiments, the amplicons are produced in a highly multiplexed, single tube amplification reaction. In some embodiments, the amplicons are produced in a collection of singleplex amplification reactions (e.g., 10 to 100, 100 to 1000, or 1000 or more reactions). In some embodiments, multiple singleplex amplification reactions (e.g., a collection of singleplex amplification reactions) are pooled. In some embodiments, the singleplex amplification reactions are performed in parallel.

Production of an amplicon panel is often associated with downstream next-generation sequencing to obtain the sequences of the amplicons of the panel. That is, the amplification is used to target the genome and provide selected regions of interest for NGS. This target enrichment focuses sequencing efforts to specific regions of a genome, thus providing a more cost-effective alternative to sequencing an entire genome and providing increased depth of coverage at the regions of interest (e.g., for improved detection of rare variation and/or lower rates of false negatives and/or false positives). Moreover, NGS provides a technology for targeting multiple amplicons in a single test.

### Methods

The technology also provides embodiments of methods for amplifying a nucleic acid, e.g., to provide an input (e.g., a NGS sequencing library; an amplicon panel library) to a NGS platform. Some embodiments of the methods comprise providing a sample comprising a polynucleotide to be sequenced. In certain exemplary embodiments, a polynucleotide (e.g., a nucleic acid sequence of interest, e.g., a target sequence, e.g., a template sequence) is at least about 1,000; 1,500; 2,000; 2,500; 3,000; 3,500; 4,000; 4,500; 5,000; 5,500; 6,000; 6,500; 7,000; 7,500; 8,000; 8,500; 9,000; 9,500; 1,000,000; 2,000,000; 3,000,000; 4,000,000; 5,000,000; 6,000,000; 7,000,000; 8,000,000; 9,000,000; 10,000,000; 15,000,000; 20,000,000; 25,000,000; 30,000,000; 35,000,000; 40,000,000; 45,000,000; 50,000,000 or more nucleotides in length. In certain aspects, a nucleic acid sequence of interest is a DNA sequence such as, e.g., a regulatory element (e.g., a promoter region, an enhancer region, a coding region, a non-coding region, and the like), a gene, a genome, a genomic gap, a DNA sequence involved in a pathway (e.g., a metabolic pathway (e.g., nucleotide metabolism, carbohydrate metabolism, amino acid metabolism, lipid metabolism, co-factor metabolism, vitamin metabolism, energy metabolism, and the like), a DNA sequence involved in a signaling pathway, a DNA sequence involved in a biosynthetic pathway, a DNA sequence involved in an immunological pathway, a developmental pathway, and the like), and the like. In yet other aspects, a nucleic acid sequence of interest is the length of a gene, e.g., between about 500 nucleotides and 5,000 nucleotides in length. In still other aspects, a nucleic acid sequence of interest is the length of a genome (e.g., a phage genome, a viral genome, a bacterial genome, a fungal genome, a plant genome, an animal genome (e.g., a human genome), or the like).

In some embodiments, a nucleic acid is fragmented to provide a polynucleotide to be sequenced. In some embodiments, fragmenting a nucleic acid comprises shearing a nucleic acid in a sample, e.g., by sonicating (e.g., sonifying) a sample comprising a nucleic acid (e.g., a sample comprising a nucleic acid to be sequenced). In some embodiments, fragmenting a nucleic acid comprises digesting with an enzyme (e.g., a restriction enzyme), nebulizing, and/or hydrodynamic shearing.

In some embodiments, a sample comprising a nucleic acid (e.g., a sample comprising one or more polynucleotides) is size-selected, e.g., to provide a polynucleotide of a preferred, defined size or within a preferred, defined range of sizes.

In some embodiments, the methods comprise amplifying a polynucleotide to be sequenced with a hairpin oligonucleotide as described herein (e.g., a hairpin oligonucleotide comprising an amplicon-specific priming sequence and an adaptor (e.g., an adaptor comprising a universal sequence (e.g., comprising a platform-dependent sequence)); e.g., a hairpin oligonucleotide comprising a loop region, a fluorescent moiety, a quencher moiety, and a blocker (e.g., exonuclease resistant) moiety). Exemplary embodiments comprise providing a hairpin oligonucleotide as described herein, a polymerase (e.g., a DNA polymerase (e.g., a polymerase comprising an exonuclease activity, e.g., a polymerase comprising a 5′ to 3′ nuclease activity or a polymerase (e.g., a high-fidelity polymerase) comprising a proof-reading activity, a 3′ exonuclease activity, and/or a strand displacement activity, but lacking a 5′ exonuclease activity)), nucleotide monomers (dNTPs), and a suitable reaction buffer; mixing the hairpin oligonucleotide, polymerase, nucleotide monomers, and reaction buffer to provide an amplification reaction mixture; thermocycling the amplification reaction mixture to produce one or more amplicons (e.g., a sequencing library or amplicon panel library); and providing the one or more amplicons (e.g., a sequencing library) as input to a NGS platform or system. Some embodiments comprise providing a second hairpin primer as described herein (e.g., a hairpin primer comprising an amplicon-specific priming sequence and an adaptor (e.g., an adaptor comprising a universal sequence (e.g., comprising a platform-dependent sequence)); e.g., a hairpin oligonucleotide comprising a loop region, a fluorescent moiety, a quencher moiety, and a blocker (e.g., exonuclease resistant) moiety). The first and/or second primers optionally comprise a tag (e.g., a tag comprising a linker, index, capture sequence, restriction site, primer binding site, antigen, and/or other functional site described herein).

In some embodiments the methods comprise sequencing a nucleic acid, e.g., using a NGS platform or system. Some embodiments comprise monitoring a signal during the amplification (e.g., a fluorescent signal), e.g., in some embodiments the method comprises a real-time quantitative amplification, e.g., in some embodiments the methods comprise quantifying an amplicon, e.g., to measure the size (e.g., the maximum size, the minimum size, the average size, the size range, etc.) of amplicons and/or to measure the concentration, number, or mass of the amplicons. In some embodiments, the quality of the library is assessed, e.g., by monitoring a fluorescent signal. Accordingly, in some embodiments the methods provided herein produce sequencing data from an individual target sequence. In some embodiments, a sample comprising an amplicon is diluted.

In some embodiments, the products of multiple (e.g., 2 or more, e.g., 3, 4, 5, 6, 7, 8, 9, 10, 15, 20, 25, 30, 35, 40, 45, 50 or more) amplification reaction mixtures are combined (e.g., mixed) to provide a multiplex library. In some embodiments, multiple libraries (e.g., from multiple subjects, samples, sources, BACs, etc.) are mixed to provide a pooled multiplexed library. Accordingly, methods provided herein comprise pooling multiple, uniquely identifiable, sample libraries that are demultiplexed in silico following sequencing (e.g., in some embodiments, the methods comprise demultiplexing sequence data, e.g., using a sequence of the index sequence to associate a sequence with its source (e.g., with a subject, sample, BAC, etc.). Accordingly, some embodiments comprise generating sequencing libraries from different samples, pooling sequencing libraries from different samples, and sequencing the pooled library in the same sequencing run. The index segments comprise characteristic sequences that are distinct for each sample.

In some embodiments, the samples are purified to remove contaminants or components from previous reactions (e.g., salts, enzymes) that may inhibit subsequent steps of the methods.

### Nucleic Acid Sequencing Platforms

In some embodiments of the technology, nucleic acid sequence data are generated. Various embodiments of nucleic acid sequencing platforms (e.g., a nucleic acid sequencers) include components as described below. According to various embodiments, a sequencing instrument includes a fluidic delivery and control unit, a sample processing unit, a signal detection unit, and a data acquisition, analysis and control unit. Various embodiments of the instrument provide for automated sequencing that is used to gather sequence information from a plurality of sequences in parallel and/or substantially simultaneously.

In some embodiments, the fluidics delivery and control unit includes a reagent delivery system. The reagent delivery system includes a reagent reservoir for the storage of various reagents. The reagents can include RNA-based primers, forward/reverse DNA primers, nucleotide mixtures (e.g., compositions comprising nucleotide analogs as provided herein) for sequencing-by-synthesis, buffers, wash reagents, blocking reagents, stripping reagents, and the like. Additionally, the reagent delivery system can include a pipetting system or a continuous flow system that connects the sample processing unit with the reagent reservoir.

In some embodiments, the sample processing unit includes a sample chamber, such as a flow cell, a substrate, a micro-array, a multi-well tray, or the like. The sample processing unit can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber. In some embodiments, the signal detection unit can include an imaging or detection sensor. For example, the imaging or detection sensor (e.g., a fluorescence detector or an electrical detector) can include a CCD, a CMOS, an ion sensor, such as an ion sensitive layer overlying a CMOS, a current detector, or the like. The signal detection unit can include an excitation system to cause a probe, such as a fluorescent dye, to emit a signal. The detection system can include an illumination source, such as arc lamp, a laser, a light emitting diode (LED), or the like. In particular embodiments, the signal detection unit includes optics for the transmission of light from an illumination source to the sample or from the sample to the imaging or detection sensor. Alternatively, the signal detection unit may not include an illumination source, such as for example, when a signal is produced spontaneously as a result of a sequencing reaction. For example, a signal can be produced by the interaction of a released moiety, such as a released ion interacting with an ion sensitive layer, or a pyrophosphate reacting with an enzyme or other catalyst to produce a chemiluminescent signal. In another example, changes in an electrical current, voltage, or resistance are detected without the need for an illumination source.

In some embodiments, a data acquisition analysis and control unit monitors various system parameters. The system parameters can include temperature of various portions of the instrument, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of the instruments and systems are used to practice sequencing methods such as sequencing by synthesis, single molecule methods, and other sequencing techniques. Sequencing by synthesis can include the incorporation of dye labeled nucleotides, chain termination, ion/proton sequencing, pyrophosphate sequencing, or the like. Single molecule techniques can include staggered sequencing, where the sequencing reaction is paused to determine the identity of the incorporated nucleotide.

In some embodiments, the sequencing instrument determines the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In some embodiments, the nucleic acid can include or be derived from a fragment library, a mate pair library, a ChIP fragment, or the like. In some embodiments, the nucleic acid can include or be derived from an amplicon library produced according to the technology provided herein. In particular embodiments, the sequencing instrument can obtain the sequence information from a single nucleic acid molecule or from a group of substantially identical nucleic acid molecules.

In some embodiments, the sequencing instrument can output nucleic acid sequencing read data in a variety of different output data file types/formats, including, but not limited to: *.txt, *.fasta, *.csfasta, *seq.txt, *qseq.txt, *.fastq, *.sff, *prb.txt, *.sms, *srs, and/or *.qv.

### Next-Generation Sequencing Technologies

Exemplary NGS platforms and system include, but are not limited to, single molecule methods and sequencing-by-synthesis methods. Particular sequencing technologies contemplated by the technology are next-generation sequencing (NGS) methods that share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), the NGS fragment library is clonally amplified in-situ by capturing single template molecules with beads bearing oligonucleotides complementary to the adapters. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 106 sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, the fragments of the NGS fragment library are captured on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 100 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves clonal amplification of the NGS fragment library by emulsion PCR. Following this, beads bearing template are immobilized on a derivatized surface of a glass flow-cell, and a primer complementary to the adapter oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, HeliScope by Helicos BioSciences is employed (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Sequencing is achieved by addition of polymerase and serial addition of fluorescently-labeled dNTP reagents. Incorporation events result in a fluor signal corresponding to the dNTP, and signal is captured by a CCD camera before each round of dNTP addition. Sequence read length ranges from 25-50 nucleotides, with overall output exceeding 1 billion nucleotide pairs per analytical run.

In some embodiments, 454 sequencing by Roche is used (Margulies et al. (2005) Nature 437: 376-380). 454 sequencing involves two steps. In the first step, DNA is sheared into fragments of approximately 300-800 base pairs and the fragments are blunt ended. Oligonucleotide adapters are then ligated to the ends of the fragments. The adapters serve as primers for amplification and sequencing of the fragments. The fragments can be attached to DNA capture beads, e.g., streptavidin-coated beads using, e.g., an adapter that contains a 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

The Ion Torrent technology is a method of DNA sequencing based on the detection of hydrogen ions that are released during the polymerization of DNA (see, e.g., Science 327(5970): 1190 (2010); U. S. Pat. Appl. Pub. Nos. 20090026082, 20090127589, 20100301398, 20100197507, 20100188073, and 20100137143, incorporated by reference in their entireties for all purposes). A microwell contains a fragment of the NGS fragment library to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs. However, the cost of acquiring a pH-mediated sequencer is approximately $50,000, excluding sample preparation equipment and a server for data analysis.

Another exemplary nucleic acid sequencing approach that may be adapted for use with the present technology was developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “HIGH THROUGHPUT NUCLEIC ACID SEQUENCING BY EXPANSION,” filed Jun. 19, 2008, which is incorporated herein in its entirety.

Other single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which fragments of the NGS fragment library are immobilized, primed, then subjected to strand extension using a fluorescently-modified polymerase and florescent acceptor molecules, resulting in detectible fluorescence resonance energy transfer (FRET) upon nucleotide addition.

Another real-time single molecule sequencing system developed by Pacific Biosciences (Voelkerding et al., Clinical Chem., 55: 641-658, 2009; MacLean et al., Nature Rev. Microbiol., 7: 287-296; U.S. Pat. No. 7,170,050; U.S. Pat. No. 7,302,146; U.S. Pat. No. 7,313,308; U.S. Pat. No. 7,476,503; all of which are herein incorporated by reference) utilizes reaction wells 50-100 nm in diameter and encompassing a reaction volume of approximately 20 zeptoliters (10⁻²¹ L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, the single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters (10⁻²¹ L). At this volume, the activity of a single molecule can be detected amongst a background of thousands of labeled nucleotides. The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations which promote enzyme speed, accuracy, and processivity. Due to the small size of the ZMW, even at these high, biologically relevant concentrations, the detection volume is occupied by nucleotides only a small fraction of the time. In addition, visits to the detection volume are fast, lasting only a few microseconds, due to the very small distance that diffusion has to carry the nucleotides. The result is a very low background.

In some embodiments, nanopore sequencing is used (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

In some embodiments, a sequencing technique uses a chemical-sensitive field effect transistor (chemFET) array to sequence DNA (for example, as described in US Patent Application Publication No. 20090026082). In one example of the technique, DNA molecules are placed into reaction chambers, and the template molecules are hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

In some embodiments, sequencing technique uses an electron microscope (Moudrianakis E. N. and Beer M. Proc Natl Acad Sci USA. 1965 March; 53:564-71). In one example of the technique, individual DNA molecules are labeled using metallic labels that are distinguishable using an electron microscope. These molecules are then stretched on a flat surface and imaged using an electron microscope to measure sequences.

In some embodiments, “four-color sequencing by synthesis using cleavable fluorescents nucleotide reversible terminators” as described in Turro, et al. PNAS 103: 19635-40 (2006) is used, e.g., as commercialized by Intelligent Bio-Systems. The technology described in U.S. Pat. Appl. Pub. Nos. 2010/0323350, 2010/0063743, 2010/0159531, 20100035253, 20100152050, incorporated herein by reference for all purposes.

Processes and systems for such real time sequencing that may be adapted for use with the technology are described in, for example, U.S. Pat. No. 7,405,281, entitled “Fluorescent nucleotide analogs and uses therefor”, issued Jul. 29, 2008 to Xu et al.; 7,315,019, entitled “Arrays of optical confinements and uses thereof”, issued Jan. 1, 2008 to Turner et al.; U.S. Pat. No. 7,313,308, entitled “Optical analysis of molecules”, issued Dec. 25, 2007 to Turner et al.; U.S. Pat. No. 7,302,146, entitled “Apparatus and method for analysis of molecules”, issued Nov. 27,2007 to Turner et al.; and 7,170,050, entitled “Apparatus and methods for optical analysis of molecules”, issued Jan. 30, 2007 to Turner et al.; and U.S. Pat. Pub. Nos. 20080212960, entitled “Methods and systems for simultaneous real-time monitoring of optical signals from multiple sources”, filed Oct. 26, 2007 by Lundquist et al.; 20080206764, entitled “Flowcell system for single molecule detection”, filed Oct. 26, 2007 by Williams et al.; 20080199932, entitled “Active surface coupled polymerases”, filed Oct. 26, 2007 by Hanzel et al.; 20080199874, entitled “CONTROLLABLE STRAND SCISSION OF MINI CIRCLE DNA”, filed Feb. 11, 2008 by Otto et al.; 20080176769, entitled “Articles having localized molecules disposed thereon and methods of producing same”, filed Oct. 26, 2007 by Rank et al.; 20080176316, entitled “Mitigation of photodamage in analytical reactions”, filed Oct. 31, 2007 by Eid et al.; 20080176241, entitled “Mitigation of photodamage in analytical reactions”, filed Oct. 31, 2007 by Eid et al.; 20080165346, entitled “Methods and systems for simultaneous real-time monitoring of optical signals from multiple sources”, filed Oct. 26, 2007 by Lundquist et al.; 20080160531, entitled “Uniform surfaces for hybrid material substrates and methods for making and using same”, filed Oct. 31, 2007 by Korlach; 20080157005, entitled “Methods and systems for simultaneous real-time monitoring of optical signals from multiple sources”, filed Oct. 26, 2007 by Lundquist et al.; 20080153100, entitled “Articles having localized molecules disposed thereon and methods of producing same”, filed Oct. 31, 2007 by Rank et al.; 20080153095, entitled “CHARGE SWITCH NUCLEOTIDES”, filed Oct. 26, 2007 by Williams et al.; 20080152281, entitled “Substrates, systems and methods for analyzing materials”, filed Oct. 31, 2007 by Lundquist et al.; 20080152280, entitled “Substrates, systems and methods for analyzing materials”, filed Oct. 31, 2007 by Lundquist et al.; 20080145278, entitled “Uniform surfaces for hybrid material substrates and methods for making and using same”, filed Oct. 31, 2007 by Korlach; 20080128627, entitled “SUBSTRATES, SYSTEMS AND METHODS FOR ANALYZING MATERIALS”, filed Aug. 31, 2007 by Lundquist et al.; 20080108082, entitled “Polymerase enzymes and reagents for enhanced nucleic acid sequencing”, filed Oct. 22, 2007 by Rank et al.; 20080095488, entitled “SUBSTRATES FOR PERFORMING ANALYTICAL REACTIONS”, filed Jun. 11, 2007 by Foquet et al.; 20080080059, entitled “MODULAR OPTICAL COMPONENTS AND SYSTEMS INCORPORATING SAME”, filed Sep. 27, 2007 by Dixon et al.; 20080050747, entitled “Articles having localized molecules disposed thereon and methods of producing and using same”, filed Aug. 14, 2007 by Korlach et al.; 20080032301, entitled “Articles having localized molecules disposed thereon and methods of producing same”, filed Mar. 29, 2007 by Rank et al.; 20080030628, entitled “Methods and systems for simultaneous real-time monitoring of optical signals from multiple sources”, filed Feb. 9, 2007 by Lundquist et al.; 20080009007, entitled “CONTROLLED INITIATION OF PRIMER EXTENSION”, filed Jun. 15,2007 by Lyle et al.; 20070238679, entitled “Articles having localized molecules disposed thereon and methods of producing same”, filed Mar. 30, 2006 by Rank et al.; 20070231804, entitled “Methods, systems and compositions for monitoring enzyme activity and applications thereof”, filed Mar. 31, 2006 by Korlach et al.; 20070206187, entitled “Methods and systems for simultaneous real-time monitoring of optical signals from multiple sources”, filed Feb. 9, 2007 by Lundquist et al.; 20070196846, entitled “Polymerases for nucleotide analog incorporation”, filed Dec. 21, 2006 by Hanzel et al.; 20070188750, entitled “Methods and systems for simultaneous real-time monitoring of optical signals from multiple sources”, filed Jul. 7, 2006 by Lundquist et al.; 20070161017, entitled “MITIGATION OF PHOTODAMAGE IN ANALYTICAL REACTIONS”, filed Dec. 1, 2006 by Eid et al.; 20070141598, entitled “Nucleotide Compositions and Uses Thereof”, filed Nov. 3, 2006 by Turner et al.; 20070134128, entitled “Uniform surfaces for hybrid material substrate and methods for making and using same”, filed Nov. 27, 2006 by Korlach; 20070128133, entitled “Mitigation of photodamage in analytical reactions”, filed Dec. 2, 2005 by Eid et al.; 20070077564, entitled “Reactive surfaces, substrates and methods of producing same”, filed Sep. 30, 2005 by Roitman et al.; 20070072196, entitled “Fluorescent nucleotide analogs and uses therefore”, filed Sep. 29, 2005 by Xu et al; and 20070036511, entitled “Methods and systems for monitoring multiple optical signals from a single source”, filed Aug. 11, 2005 by Lundquist et al.; and Korlach et al. (2008) “Selective aluminum passivation for targeted immobilization of single DNA polymerase molecules in zero-mode waveguide nanostructures” PNAS 105(4): 1176-81, all of which are herein incorporated by reference in their entireties.

In some embodiments, the quality of data produced by a next-generation sequencing platform depends on the concentration of DNA (e.g., an amplicon panel library) that is loaded onto the sequencer workflow clonal amplification step. For instance, loading a concentration that is below a minimal threshold may result in low or sub-optimal sequencer output while loading a concentration that is above a maximum threshold may result in low quality sequence or no sequencer output. Accordingly, the technology provided herein finds use in preparing a sample having an appropriate concentration for sequencing, e.g., such that the sequence data that is output has a desirable quality.

### Nucleic Acid Sequence Analysis

In some embodiments, a computer-based analysis program is used to translate the raw data generated by the detection assay (e.g., sequencing reads) into data of predictive value for an end user (e.g., medical personnel). The user can access the predictive data using any suitable means. Thus, in some preferred embodiments, the present technology provides the further benefit that the user, who is not likely to be trained in genetics or molecular biology, need not understand the raw data. The data is presented directly to the end user in a useful form. The user is then able to immediately utilize the information to determine useful information (e.g., in medical diagnostics, research, or screening).

Some embodiments provide a system for reconstructing a nucleic acid sequence. The system can include a nucleic acid sequencer, a sample sequence data storage, a reference sequence data storage, and an analytics computing device/server/node. In some embodiments, the analytics computing device/server/node can be a workstation, mainframe computer, personal computer, mobile device, etc. The nucleic acid sequencer can be configured to analyze (e.g., interrogate) a nucleic acid fragment (e.g., single fragment, mate-pair fragment, paired-end fragment, etc.) utilizing all available varieties of techniques, platforms, or technologies to obtain nucleic acid sequence information, in particular the methods as described herein using compositions provided herein. In some embodiments, the nucleic acid sequencer is in communications with the sample sequence data storage either directly via a data cable (e.g., serial cable, direct cable connection, etc.) or bus linkage or, alternatively, through a network connection (e.g., Internet, LAN, WAN, VPN, etc.). In some embodiments, the network connection can be a “hardwired” physical connection. For example, the nucleic acid sequencer can be communicatively connected (via Category 5 (CATS), fiber optic, or equivalent cabling) to a data server that is communicatively connected (via CATS, fiber optic, or equivalent cabling) through the Internet and to the sample sequence data storage. In some embodiments, the network connection is a wireless network connection (e.g., Wi-Fi, WLAN, etc.), for example, utilizing an 802.11 a/b/g/n or equivalent transmission format. In practice, the network connection utilized is dependent upon the particular requirements of the system. In some embodiments, the sample sequence data storage is an integrated part of the nucleic acid sequencer.

In some embodiments, the sample sequence data storage is any database storage device, system, or implementation (e.g., data storage partition, etc.) that is configured to organize and store nucleic acid sequence read data generated by nucleic acid sequencer such that the data can be searched and retrieved manually (e.g., by a database administrator or client operator) or automatically by way of a computer program, application, or software script. In some embodiments, the reference data storage can be any database device, storage system, or implementation (e.g., data storage partition, etc.) that is configured to organize and store reference sequences (e.g., whole or partial genome, whole or partial exome, SNP, gen, etc.) such that the data can be searched and retrieved manually (e.g., by a database administrator or client operator) or automatically by way of a computer program, application, and/or software script. In some embodiments, the sample nucleic acid sequencing read data can be stored on the sample sequence data storage and/or the reference data storage in a variety of different data file types/formats, including, but not limited to: *.txt, *.fasta, *.csfasta, *seq.txt, *qseq.txt, *.fastq, *.sff, *prb.txt, *.sms, *srs, and/or *.qv.

In some embodiments, the sample sequence data storage and the reference data storage are independent standalone devices/systems or implemented on different devices. In some embodiments, the sample sequence data storage and the reference data storage are implemented on the same device/system. In some embodiments, the sample sequence data storage and/or the reference data storage can be implemented on the analytics computing device/server/node. The analytics computing device/server/node can be in communications with the sample sequence data storage and the reference data storage either directly via a data cable (e.g., serial cable, direct cable connection, etc.) or bus linkage or, alternatively, through a network connection (e.g., Internet, LAN, WAN, VPN, etc.). In some embodiments, analytics computing device/server/node can host a reference mapping engine, a de novo mapping module, and/or a tertiary analysis engine. In some embodiments, the reference mapping engine can be configured to obtain sample nucleic acid sequence reads from the sample data storage and map them against one or more reference sequences obtained from the reference data storage to assemble the reads into a sequence that is similar but not necessarily identical to the reference sequence using all varieties of reference mapping/alignment techniques and methods. The reassembled sequence can then be further analyzed by one or more optional tertiary analysis engines to identify differences in the genetic makeup (genotype), gene expression or epigenetic status of individuals that can result in large differences in physical characteristics (phenotype). For example, in some embodiments, the tertiary analysis engine can be configured to identify various genomic variants (in the assembled sequence) due to mutations, recombination/crossover, or genetic drift. Examples of types of genomic variants include, but are not limited to: single nucleotide polymorphisms (SNPs), copy number variations (CNVs), insertions/deletions (Indels), inversions, etc. The optional de novo mapping module can be configured to assemble sample nucleic acid sequence reads from the sample data storage into new and previously unknown sequences. It should be understood, however, that the various engines and modules hosted on the analytics computing device/server/node can be combined or collapsed into a single engine or module, depending on the requirements of the particular application or system architecture. Moreover, in some embodiments, the analytics computing device/server/node can host additional engines or modules as needed by the particular application or system architecture.

In some embodiments, the mapping and/or tertiary analysis engines are configured to process the nucleic acid and/or reference sequence reads in color space. In some embodiments, the mapping and/or tertiary analysis engines are configured to process the nucleic acid and/or reference sequence reads in base space. It should be understood, however, that the mapping and/or tertiary analysis engines disclosed herein can process or analyze nucleic acid sequence data in any schema or format as long as the schema or format can convey the base identity and position of the nucleic acid sequence.

In some embodiments, the sample nucleic acid sequencing read and referenced sequence data can be supplied to the analytics computing device/server/node in a variety of different input data file types/formats, including, but not limited to: *.txt, *.fasta, *.csfasta, *seq.txt, *qseq.txt, *.fastq, *.sff, *prb.txt, *.sms, *srs, and/or *.qv.

Furthermore, a client terminal can be a thin client or thick client computing device. In some embodiments, a client terminal can have a web browser that can be used to control the operation of the reference mapping engine, the de novo mapping module, and/or the tertiary analysis engine. That is, the client terminal can access the reference mapping engine, the de novo mapping module, and/or the tertiary analysis engine using a browser to control their function. For example, the client terminal can be used to configure the operating parameters (e.g., mismatch constraint, quality value thresholds, etc.) of the various engines, depending on the requirements of the particular application. Similarly, a client terminal can also display the results of the analysis performed by the reference mapping engine, the de novo mapping module, and/or the tertiary analysis engine.

The present technology also encompasses any method capable of receiving, processing, and transmitting the information to and from laboratories conducting the assays, information provides, medical personal, and subjects.

### Kits

Some embodiments provide kits for producing a sequencing library (e.g., an amplicon library). For example, kit embodiments comprise components such as one or more hairpin oligonucleotides as described herein, dNTP monomers (e.g., dATP, dCTP, dGTP, and dTTP), a polymerase (e.g., a DNA polymerase comprising exonuclease (e.g., 5′ to 3′ exonuclease) activity or a polymerase (e.g., a high-fidelity polymerase) comprising a proof-reading activity, a 3′ exonuclease activity, and/or a strand displacement activity, but lacking a 5′ exonuclease activity), a control template, a reaction buffer, packaged in any combination. In some embodiments individual hairpin oligonucleotides of the one or more hairpin oligonucleotides comprise an adaptor (e.g., comprising a tag (e.g., comprising an index) and/or comprising a universal, platform-dependent sequence) and an amplicon-specific (e.g., target-specific) sequence. Components are provided, in some embodiments, in ready-to-use form, as a lyophilized form, in a concentrated form to be diluted for use, etc.

### Systems

The technology includes embodiments of systems comprising various components such as, e.g., reaction mixtures comprising one or more hairpin oligonucleotides, e.g., as described herein, a thermocycling apparatus, and a computer-based analysis program, e.g., as described herein. Some embodiments of systems comprise a fluorescence detector, e.g., to monitor the progress of and/or quantify an amplification reaction. Embodiments of systems comprise, in various combinations of (e.g., having some or all of), one or more hairpin oligonucleotides (comprising a fluorescent moiety and a quenching moiety on the same strand of a double-stranded duplex), an amplicon library, (e.g., a multiplex amplicon library, e.g., as described herein), a NGS sequencing apparatus (including components related to the NGS sequencing workflow), and one or more reporting functionalities for providing information (e.g., sequence data) to a user in a user-readable and/or computer-readable format.

Although the disclosure herein refers to certain illustrated embodiments, it is to be understood that these embodiments are presented by way of example and not by way of limitation.

### EXAMPLES

### Example 1

### Design of Oligonucleotides

During the development of embodiments of the technology provided herein, hairpin oligonucleotides were designed to amplify a region of the human chromosome 7 (epidermal growth factor receptor (EGFR) gene) and a region of human chromosome 1 (a non-coding region of chromosome 1) (Table 1). The oligonucleotides in Table 1 named “F_egfr_trP1” (SEQ ID NO: 1) and “R_egfr_b1_A” (SEQ ID NO: 2) targeted chromosome 7 (at the EGFR gene); the oligonucleotides in Table 1 named “F_chr1_trP1” (SEQ ID NO: 3) and “R_chr1_b1_A” (SEQ ID NO: 4) targeted chromosome 1.

TABLE 1
oligonucleotide sequences and structures

| | | SEQ |
|---|---|---|
| | | ID |
| name | sequence (5′ to 3′) | NO: |
| F_egfr_ | pTCA CCG ACT GCC CAT AGA GAG GAA | 1 |
| trP1 | AGC G*c*c tcC GCT TTC CTC TCT | |
| | ATG GGC AGT CGG TGA TCC TTC | |
| | CTT TCA TGC TCT CTT CC | |
| R_egfr_ | pCTG AGT CGG AGA CAC GCA GGG ATG | 2 |
| b1_A | A*c*c atc TCA TCC CTG CGT GTC | |
| | TCC GAC TCA GCT AAG GTA ACG ATC | |
| | TTC CTC CAT CTC ATA GCT GTC | |
| F_chr1_ | pTCA CCG ACT GCC CAT AGA GAG GAA | 3 |
| trP1 | AGC G*c*c tcC GCT TTC CTC TCT | |
| | ATG GGC AGT CGG TGA TCCA AGT | |
| | CTG AAT GAG GTC TGA TG | |
| R_chr1_ | pCTG AGT CGG AGA CAC GCA GGG ATG | 4 |
| b1_A | A*c*c atc TCA TCC CTG CGT GTC | |
| | TCC GAC TCA GCT AAG GTA ACG ATTG | |
| | TGT CTA ATC AAC TGG AGA CG | |

In Table 1, the sequences in bold typeface and capital letters represent target-specific priming sequences; sequences in non-bold capital letters represent the “universal” sequences that are used subsequent to PCR for clonal amplification (e.g., for sequencing). Sequences underlined in the reverse primers (e.g., with names beginning “R_”) represent barcode/index sequences; sequences in lower case letters represent the loop region formed as a result of intra-molecular hybridization. In Table 1, an asterisk (“*”) indicates a phosphorothioate bond and a “p” indicates a phosphate group (e.g., a phosphate group from a typical oligonucleotide synthesis).

The secondary structures of the F_egfr_trP1, R_egfr_b1_A, F_chr1_trP1, and R_chr1_b1_A oligonucleotides were modeled using software (UNAFold and mFOLD, Rensselaer Polytechnic Institute) (FIG. 4A, FIG. 4B, FIG. 4C, and FIG. 4D, respectively). The modeling indicates that the oligonucleotides form stem-loop (“hairpin”) structures (FIG. 4A, FIG. 4B, FIG. 4C, and FIG. 4D).

Further, the formation of these structures is predicted to be thermodynamically favorable (e.g., having negative free energies of formation (ΔG)) at the indicated temperatures of 70° C., 62° C., and 55° C. Thermodynamic free energies (ΔG in kcal/mol) were calculated from the models using a Na ion (Na+) concentration of 60 mM, a Mg ion (Mg++) concentration of 4 mM, and at temperatures of 55° C., 62° C., and 70° C. (FIG. 4; Table 2).

TABLE 2
free energies of duplex formation

| | temperature | ΔG |
|---|---|---|
| name | ° C. | kcal/mol |
| F_egfr_trP1 | 70 | −12.40 |
| | 62 | −17.37 |
| | 55 | −21.72 |
| R_egfr_b1_A | 70 | −10.95 |
| | 62 | −15.42 |
| | 55 | −19.33 |
| F_chr1_trP1 | 70 | −12.40 |
| | 62 | −17.37 |
| | 55 | −21.72 |
| R_chr1_b1_A | 70 | −10.95 |
| | 62 | −15.42 |
| | 55 | −19.33 |

### Example 2

### Use of Oligonucleotides in Real-Time Amplification

During the development of embodiments of the technology provided herein, experiments were conducted to test exemplary hairpin oligonucleotides designed according to the technology described herein. In particular, the exemplary hairpin oligonucleotides described in Example 1 were tested in a two-plex (e.g., simultaneous detection of two targets in the same reaction) amplification using fluorescently labeled detection probes (Table 3).

TABLE 3
probes used for fluorescence detection

| | | | | SEQ |
|---|---|---|---|---|
| | | Tm | GC | ID |
| name | sequence (5′ to 3′) | (°C.) | (%) | NO: |
| EGFR | FAM-TTATGTGGTGACAG | 69.5 | 50 | 5 |
| probe | ATCACGGCTCGT-BHQ1 | | | |
| Chr1 | VIC-ACCAAACTTAGGAA | 70.3 | 50 | 6 |
| probe | CTTGCCTGCCCT-BHQ1 | | | |

The EGFR probe was labeled with a fluorescent moiety (FAM) on its 5′ end a quencher moiety (BHQ1) on its 3′ end; similarly, the Chr1 probe was labeled with a fluorescent moiety (VIC) on its 5′ end and a quencher moiety (BHQ1) on its 3′ end.

Amplification mixtures contained 1× PCR buffer, 52.5 mM Tris-HCl, 4 mM MgCl₂, 0.8 mM dNTP, 0.5 μM of each oligonucleotide primer (F_egfr_trP1, R_egfr_b1_A, F_chr1_trP1, and R_chr1_b1_A), 0.2 μM of each probe (EGFR probe and Chr1 probe), 0.6 μM of ROX dye, and 11 units of Taq polymerase (Taq gold) in a 50-μL, final reaction volume. 20 ng of purified genomic DNA was used as sample input for template.

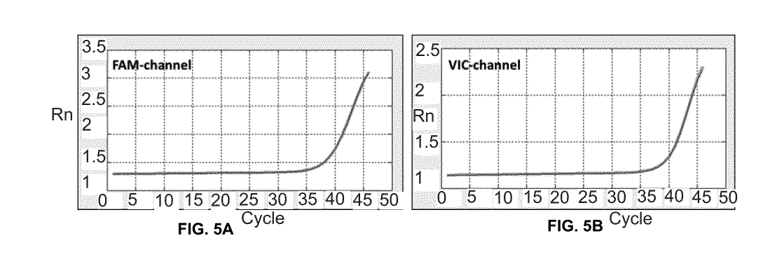

Real-time PCR cycling was performed using a temperature cycling profile as follows: 94° C. for 10 minutes; 4 cycles of 92° C. for 30 seconds, 60° C. for 30 seconds; 46 cycles of 92° C. for 30 seconds, 62° C. for 30 seconds, 58° C. for 40 seconds. After each of the 46 cycles, samples were excited with an appropriate energy source and the fluorescent emission signals were acquired. Data collected from the real-time amplification (FIG. 5) showed that both sets of oligonucleotide primers targeting chromosome 7 (FIG. 5A) and chromosome 1 (FIG. 5B) generated target-specific products (e.g., amplicons) that accumulated as expected in the reactions during amplification.

### Example 3

### Nucleic Acid Fragment Size Analysis

During the development of the technology provided herein, amplification (e.g., PCR) was performed using hairpin oligonucleotide primers (e.g., as described in Example 1) and the amplification products were analyzed to determine their size distributions (e.g., using a Bioanalyzer 2100 system (Agilent Technologies)). Amplification was performed as described in Example 2, except the reaction mixtures did not contain the real-time PCR components, probes, and ROX dye. An Agilent High-Sensitivity DNA chip was used to determine the sizes of the amplification products generated.

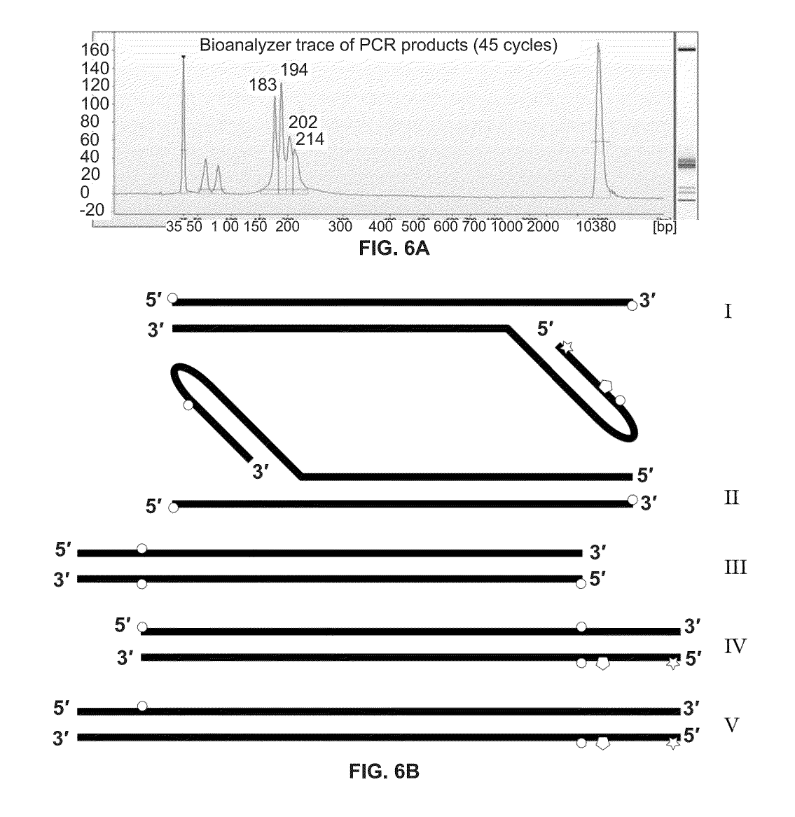

After multiple amplification cycles, the amplification was expected to produce a heterogeneous population of products having different sizes. For the particular oligonucleotide primers and templates used in this example, exemplary (e.g., predominant) intermediate products and/or end point products of approximately 176 by (see, e.g., FIG. 6B, forms I and II), 200 bp (see, e.g., FIG. 6B, form III), 203 bp (see, e.g., FIG. 6B, form IV), and 227 bp (see, e.g., FIG. 6B, form V) were expected for the EGFR (chromosome 7) amplification and products of approximately 191 bp (see, e.g., FIG. 6B, forms I and II), 215 bp (see, e.g., FIG. 6B, form III), 218 bp (see, e.g., FIG. 6B, form IV), and 242 bp (see, e.g., FIG. 6, form V) were expected for the chromosome 1 amplification. The predicted sizes of expected products were compared to the experimentally measured sizes of approximately 183 bp, 194 bp, 202 bp, and 214 bp (FIG. 6A). The experimentally measured fragment sizes (FIG. 6A) agreed with the prediction that the reaction would comprise a heterologous population of products having various sizes (FIG. 6B).

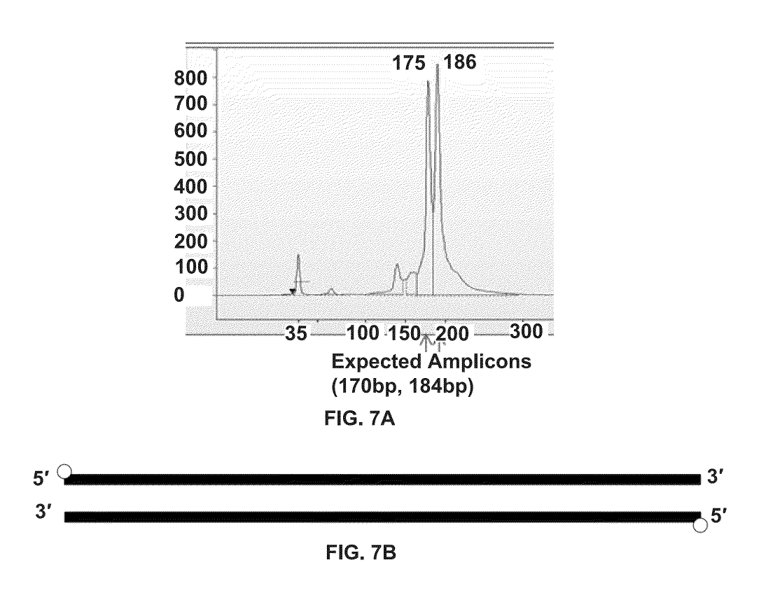

After amplification, the amplification products were treated with enzymes to convert (e.g., to fill in single-strand regions, to remove unresolved hairpin structures, etc.) the heterogeneous population of amplicons (see, e.g., FIG. 6) into a more homogenous population of products (compare, e.g., FIG. 7B with FIG. 6B). The predicted sizes of the EGFR and chromosome 1 products after enzymatic treatment (e.g., see FIG. 7B) are 176 by and 191 bp, respectively. The amplification products were treated with lambda exonuclease and Klenow DNA polymerase for 20 minutes at 37° C. After treatment, fragment analysis was performed on the products. The data collected show that the enzymatic treatment converted the heterologous amplification products for the EGFR and chromosome 1 amplifications into a final single amplicon form for each target in the two-plex reaction (FIG. 7A). After conversion, the samples comprised EGFR and chromosome 1 amplification products predominantly in the 176-bp and 191-bp forms, respectively (FIG. 7A). These forms are the double-stranded linear forms having defined ends as shown in the schematic of FIG. 7B.

### Example 4

### Production of NGS Amplicon Libraries

During the development of embodiments of the technology provided herein, experiments were conducted to compare hairpin oligonucleotide primers as described herein with existing fusion oligonucleotide technologies for producing NGS amplicon libraries. Hairpin oligonucleotide primers as described herein were designed and synthesized (F_egfr_trP1, R_egfr_b1_A, R_egfr_trP1, and F_egfr_b1_A) (Table 4).

In addition, standard fusion oligonucleotide primers (Ion Torrent fusion primers) were designed and synthesized (Table 5) to amplify the same target region as the hairpin oligonucleotides. Both types of oligonucleotide primers were used to generate amplicons for NGS (e.g., using a Life Technologies Ion Torrent PGM sequencer).

TABLE 4
oligonucleotides for NGS amplicon libraries

| | | SEQ |
|---|---|---|
| | | ID |
| name | sequence (5′ to 3′) | NO: |
| F_egfr_ | pTCA CCG ACT GCC CAT AGA GAG GAA | 1 |
| trP1 | AGC G*C*C TCC GCT TTC CTC TCT ATG | |
| | GGC AGT CGG TGA TCC TTC CTT TCA | |
| | TGC TCT CTT CC | |
| R_egfr_ | pCTG AGT CGG AGA CAC GCA GGG ATG | 2 |
| b1_A | A*C*C ATC TCA TCC CTG CGT GTC TCC | |
| | GAC TCA GCT AAG GTA ACG ATC TTC | |
| | CTC CAT CTC ATA GCT GTC | |
| R_egfr_ | pTCA CCG ACT GCC CAT AGA GAG GAA | 7 |
| trP1 | AGC G*C*C TCC GCT TTC CTC TCT ATG | |
| | GGC AGT CGG TGA TC TTC CTC CAT | |
| | CTC ATA GCT GTC | |
| F_egfr_ | pCTG AGT CGG AGA CAC GCA GGG ATG | 8 |
| b1_A | A*C*C ATC TCA TCC CTG CGT GTC TCC | |
| | GAC TCA GCT AAG GTA ACG ATCC TTC | |
| | CTT TCA TGC TCT CTT CC | |
| F_chr1_ | pTCA CCG ACT GCC CAT AGA GAG GAA | 3 |
| trP1 | AGC G*C*C TCC GCT TTC CTC TCT ATG | |
| | GGC AGT CGG TGA TCCA AGT CTG AAT | |
| | GAG GTC TGA TG | |
| R_chr1_ | pCTG AGT CGG AGA CAC GCA GGG ATG | 4 |
| b1_A | A*C*C ATC TCA TCC CTG CGT GTC TCC | |
| | GAC TCA GCT AAG GTA ACG ATTG TGT | |
| | CTA ATC AAC TGG AGA CG | |
| R_chr1_ | pTCA CCG ACT GCC CAT AGA GAG GAA | 9 |
| trP1 | AGC G*C*C TCC GCT TTC CTC TCT | |
| | ATG GGC AGT CGG TGA TTG TGT CTA | |
| | ATC AAC TGG AGA CG | |
| F_chr1_ | pCTG AGT CGG AGA CAC GCA GGG ATG | 10 |
| b1_A | A*C*C ATC TCA TCC CTG CGT GTC | |
| | TCC GAC TCA GCT AAG GTA ACG ATCCA | |
| | AGT CTG AAT GAG GTC TGA TG | |

In Table 4, an asterisk (“*”) indicates a phosphorothioate bond and a “p” indicates a phosphate group (e.g., a phosphate group from a typical oligonucleotide synthesis).

TABLE 5
standard oligonucleotides for
NGS amplicon libraries

| Ion | | |
|---|---|---|
| Torrent | | |
| fusion | | SEQ |
| primer | | ID |
| name | sequence (5′ to 3′) | NO: |
| ION-F_ | C CTC TCT ATG GGC AGT CGG | 11 |
| egfr_ | TGA TCATCACC TTC CTT | |
| trP1 | TCA TGC TCT CTT C | |
| ION-R_ | CC ATC TCA TCC CTG CGT GTC TCC | 12 |
| egfr_ | GAC TCA GCT AAG GTA ACG ATTC | |
| b1_A | TTC CTC CAT CTC ATA GCT GTCG | |
| ION-R_ | C CTC TCT ATG GGC AGT CGG | 13 |
| egfr_ | TGA TTC TTC CTC CAT CTC | |
| trP1 | ATA GCT GTCG | |
| ION-F_ | CC ATC TCA TCC CTG CGT GTC TCC | 14 |
| egfr_ | GAC TCA GCT AAG GTA ACG ATCATCACC | |
| b1_A | TTC CTT TCA TGC TCT CTT CC | |
| ION-F_ | C CTC TCT ATG GGC AGT CGG TGA | 15 |
| chr1_ | TCGCCA AGT CTG AAT GAG | |
| trP1 | GTC TGA TGA | |
| ION-R_ | CC ATC TCA TCC CTG CGT GTC | 16 |
| chr1_ | TCC GAC TCA GCT AAG GTA ACG | |
| b1_A | ATAGGCTG TGT CTA ATC AAC | |
| | TGG AGA CG | |
| ION-R_ | C CTC TCT ATG GGC AGT CGG | 17 |
| chr1_ | TGA TAGGCTG TGT CTA ATC | |
| trP1 | AAC TGG AGA CG | |
| ION-F_ | CC ATC TCA TCC CTG CGT GTC | 18 |
| chr1_ | TCC GAC TCA GCT AAG GTA ACG | |
| b1_A | ATCGCCA AGT CTG AAT GAG GTC | |
| | TGA TGA | |

To compare the hairpin oligonucleotide primers as provided by the technology described herein with the standard oligonucleotide fusion primers, four-plex amplification reactions were performed using the hairpin oligonucleotide primers (Table 4). Amplification reaction mixtures were mixed with the following components, provided here as final concentrations in the reaction mixtures: 1× PCR buffer, 52.5 mM Tris-HCl, 4 mM MgCl2, 0.8 mM dNTP, 0.25 μM of each hairpin oligonucleotide primer, and 15 units of Taq polymerase (e.g., Taq gold) in a 50-0, final reaction volume. 20 ng of purified genomic DNA was used as sample input for the template. Amplification reaction cycling was performed using the following temperature cycling profile: 95° C. for 10 minutes; 40 cycles of 95° C. for 20 seconds, 70° C. for 5 seconds, 57° C. for 45 seconds, 62° C. for 45 seconds. After amplification, the amplification products were treated with lambda exonuclease and Klenow DNA polymerase for 20 minutes at 37° C.

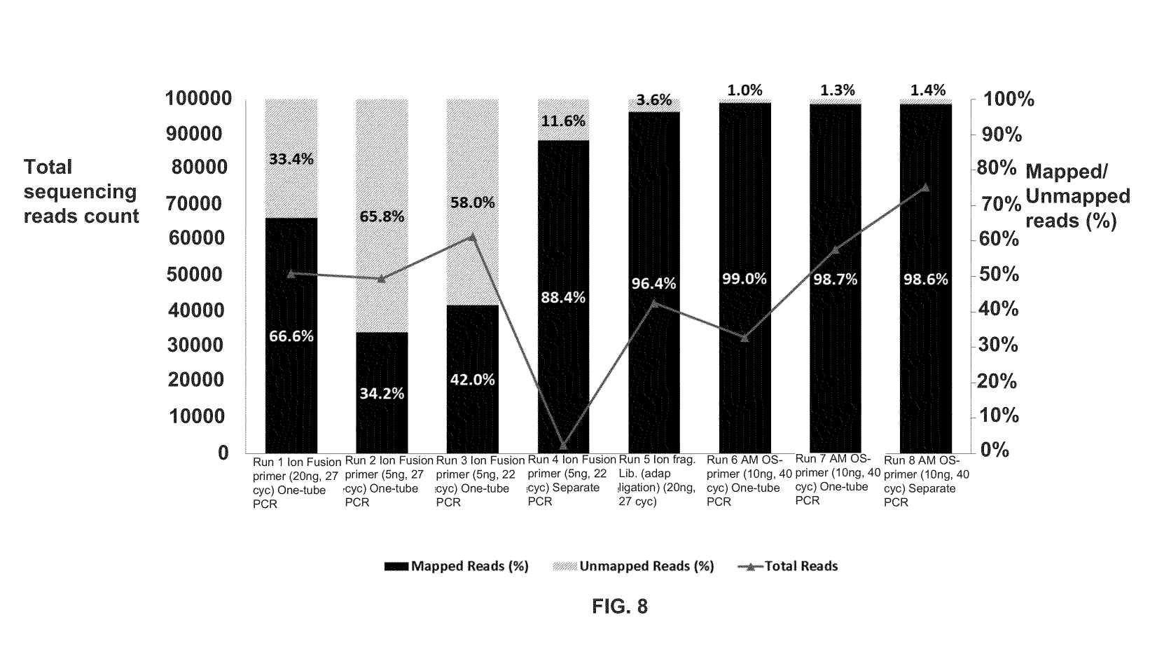

Parallel four-plex amplification reactions were performed using the standard fusion oligonucleotide primers (Ion Torrent fusion primers). These reactions using the standard fusion oligonucleotide primers used the same reaction conditions as noted above for the hairpin oligonucleotide primers except for minor changes in the temperature cycling as follows: 94° C. for 10 minutes; 4 cycles of 92° C. for 30 seconds, 60° C. for 30 seconds; 23 cycles of 92° C. for 30 seconds, 62° C. for 30 seconds, 58° C. for 40 seconds. Both hairpin oligonucleotide primer and standard fusion oligonucleotide primer NGS libraries were clonally amplified on beads (e.g., using Life Technologies One-Touch machines (ePCR)) and subsequently enriched (e.g., on Enrichment Stations) prior to sequencing (e.g., on an Ion Torrent PGM sequencer). Multiple runs representing libraries produced under different library generation conditions were processed on the sequencer and the performance of library generation was assessed by comparing sequence mapping efficiencies (FIG. 8). The data collected demonstrate that amplicon libraries generated with the standard fusion primers (FIG. 8, columns labeled “Ion Fusion Primer”) resulted in a higher number of unmapped reads than the libraries generated with the hairpin oligonucleotide primers (FIG. 8, columns labeled “AM OS-primer”) or with standard adaptor ligation methods (FIG. 8, column labeled “Ion frag. Lib. (adap ligation)”). In particular, the libraries generated from fusion primer methods produced sequences with mapped/unmapped reads (in percentages) of 66.6/33.4, 34.2/65.8, 42.0/58.0, and 88.4/11.6; the libraries produced by adaptor ligation methods produced sequences with mapped/unmapped reads (in percentages) of 96.4/3.6; and the libraries generated from the hairpin primers and associated methods as described herein produced sequences with mapped/unmapped reads (in percentages) of 99.0/1.0, 98.7/1.3, and 98.6/1.4 (FIG. 8).

### Example 5

### Detection of Copy Number Variation

During the development of the technology provided herein, experiments were conducted in which the technology was used to determine copy number variation (CNV) in test samples. The test samples were two purified genomic DNA samples (sample 384 and sample 356) derived from glioblastoma tumor tissue and having a DNA copy number status previously determined by fluorescent in situ hybridization of the EGFR gene. Sample 384 had greater than 5× amplification of the EGFR gene and sample 356 had no amplification of the EGFR gene.

Hairpin oligonucleotide primers were designed and synthesized to generate NGS amplicon libraries for bi-directional DNA sequencing (e.g., using a Life Technologies Ion Torrent PGM sequencer apparatus). Barcode sequences were introduced to enable multiplexed sequencing of both samples and subsequent demultiplexing or deconvolution of sequence read data from the multiplex sequencing. In Table 6, b1 signifies an oligonucleotide comprising barcode sequence number 1 (“barcode1”) and b3 signifies an oligonucleotide comprising barcode sequence number 3 (“barcode3”).

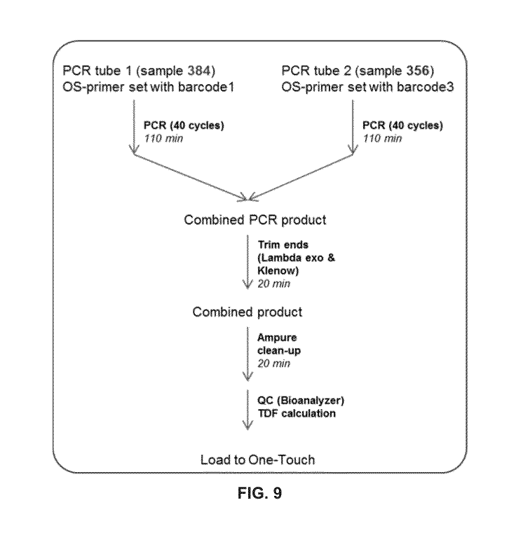

To prepare amplicon libraries, two amplification reactions were prepared in parallel then mixed (see, e.g., FIG. 9). In the first reaction, hairpin oligonucleotide primers comprising a first bar code (barcode1) were used to prepare a first amplicon library from sample 384. In the second reaction, hairpin oligonucleotide primers comprising a second barcode (barcode3) were used to prepare a second amplicon library from sample 356. 40 temperature cycles were used for both amplification reactions (taking a time of approximately 110 minutes). The products of these two amplifications were combined to provide a sample comprising a combined pool of amplification products. The combined amplification products were treated with lambda exonuclease and Klenow DNA polymerase for 20 minutes at 37° C., then cleaned-up (e.g., with Ampure beads) to remove unincorporated nucleotides, primers, etc. The cleaned-up sample was assessed (e.g., using a Bioanalyzer 2100 (Agilent Technologies)) for quality and fragment size distribution prior to introducing the sample into the sequencing workflow for clonal amplification on beads (e.g., using Life Technologies One-Touch machines (ePCR)). The hairpin oligonucleotide primer amplicon libraries were clonally amplified (e.g., on beads using a Life Technologies One-Touch apparatus (ePCR)) and subsequently enriched (e.g., on Enrichment Stations) prior to sequencing (e.g., on an Ion Torrent PGM sequencer apparatus).

TABLE 6
hairpin oligonucleotides
for analysis of CNV

| | | SEQ |
|---|---|---|
| | | ID |
| name | sequence (5′ to 3′) | NO: |
| F_ | pTCA CCG ACT GCC CAT AGA GAG GAA | 1 |
| egfr_ | AGC G*C*C TCC GCT TTC CTC TCT | |
| trP1 | ATG GGC AGT CGG TGA TCC TTC | |
| | CTT TCA TGC TCT CTT CC | |
| R_ | pCTG AGT CGG AGA CAC GCA GGG ATG | 2 |
| egfr_ | A*C*C ATC TCA TCC CTG CGT GTC | |
| b1_A | TCC GAC TCA GCT AAG GTA ACG ATC | |
| | TTC CTC CAT CTC ATA GCT GTC | |
| R_ | pTCA CCG ACT GCC CAT AGA GAG GAA | 7 |
| egfr_ | AGC G*C*C TCC GCT TTC CTC TCT ATG | |
| trP1 | GGC AGT CGG TGA TC TTC CTC CAT | |
| | CTC ATA GCT GTC | |
| F_ | pCTG AGT CGG AGA CAC GCA GGG ATG | 8 |
| egfr_ | A*C*C ATC TCA TCC CTG CGT GTC | |
| b1_A | TCC GAC TCA GCT AAG GTA ACG ATCC | |
| | TTC CTT TCA TGC TCT CTT CC | |
| F_ | pTCA CCG ACT GCC CAT AGA GAG GAA | 3 |
| chr1_ | AGC G*C*C TCC GCT TTC CTC TCT | |
| trP1 | ATG GGC AGT CGG TGA TCCA AGT | |
| | CTG AAT GAG GTC TGA TG | |
| R_ | pCTG AGT CGG AGA CAC GCA GGG ATG | 4 |
| chr1_ | A*C*C ATC TCA TCC CTG CGT GTC | |
| b1_A | TCC GAC TCA GCT AAG GTA ACG ATTG | |
| | TGT CTA ATC AAC TGG AGA CG | |
| R_ | pTCA CCG ACT GCC CAT AGA GAG GAA | 9 |
| chr1_ | AGC G*C*C TCC GCT TTC CTC TCT | |
| trP1 | ATG GGC AGT CGG TGA TTG TGT | |
| | CTA ATCAAC TGG AGA CG | |
| F_ | pCTG AGT CGG AGA CAC GCA GGG ATG | 10 |
| chr1_ | A*C*C ATC TCA TCC CTG CGT GTC TCC | |
| b1_A | GAC TCA GCT AAG GTA ACG ATCCA AGT | |
| | CTG AAT GAG GTC TGA TG | |
| R_ | pCTG AGT CGG AGA CAC GCA GGG ATG | 19 |
| egfr_ | A*C*C ATC TCA TCC CTG CGT GTC | |
| b3_A | TCC GAC TCA GAA GAG GAT TCG ATC | |
| | TTC CTC CAT CTC ATA GCT GTC | |
| F_egfr_ | pCTG AGT CGG AGA CAC GCA GGG ATG | 20 |
| b3_A | A*C*C ATC TCA TCC CTG CGT GTC | |
| | TCC GAC TCA GAA GAG GAT TCG | |
| | ATCC TTC CTT TCA TGC TCT CTT CC | |
| R_chr1_ | pCTG AGT CGG AGA CAC GCA GGG ATG | 21 |
| b3_A | A*C*C ATC TCA TCC CTG CGT GTC | |
| | TCC GAC TCA GAA GAG GAT TCG | |
| | ATTG TGT CTA ATC AAC TGG AGA CG | |
| F_chr1_ | pCTG AGT CGG AGA CAC GCA GGG ATG | 22 |
| b3_A | A*C*C ATC TCA TCC CTG CGT GTC | |
| | TCC GAC TCA GAA GAG GATTCG ATCCA | |
| | AGT CTG AAT GAG GTC TGA TG | |

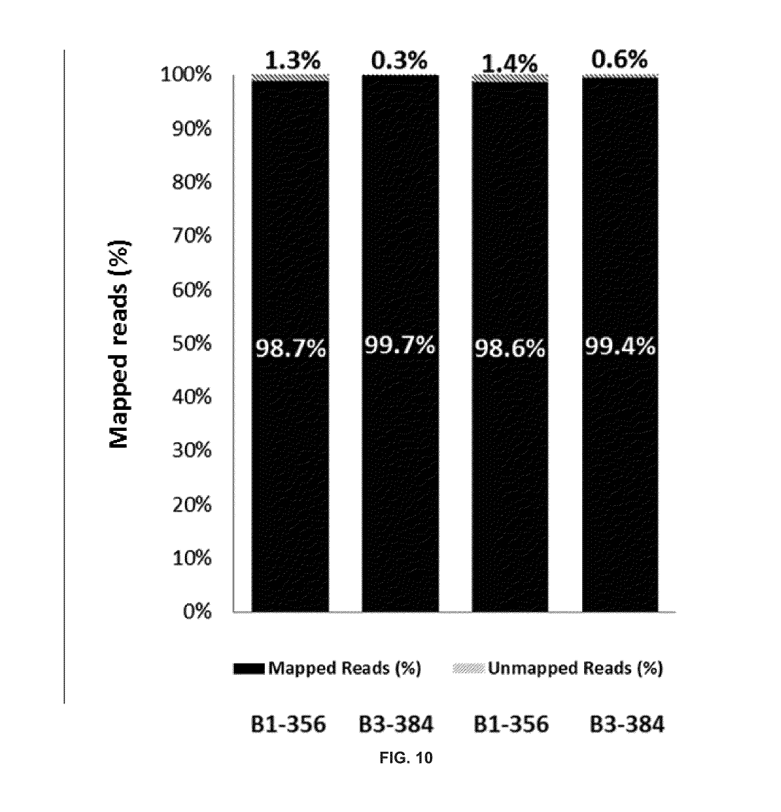

Two multiplexed runs prepared from different library generation conditions were tested. In particular, one-tube multiplex amplification (“Run 1”) was compared with pooling of multiple, separate single-plex amplifications (“Run 2”). The performance of library generation was first assessed by comparing sequence mapping efficiencies. The data indicated that greater than 98.5% of raw reads were mapped to reference sequences for all runs (FIG. 10). In FIG. 10, column 1 shows mapped and unmapped reads for both Run 1 and Run 2 of sample B1-356, column 2 shows mapped and unmapped reads for both Run 1 and Run 2 of sample B3-384, column 3 shows mapped and unmapped reads for both Run 1 and Run 2 of sample B1-356, and column 4 shows mapped and unmapped reads for both Run 1 and Run 2 for sample B3-384.

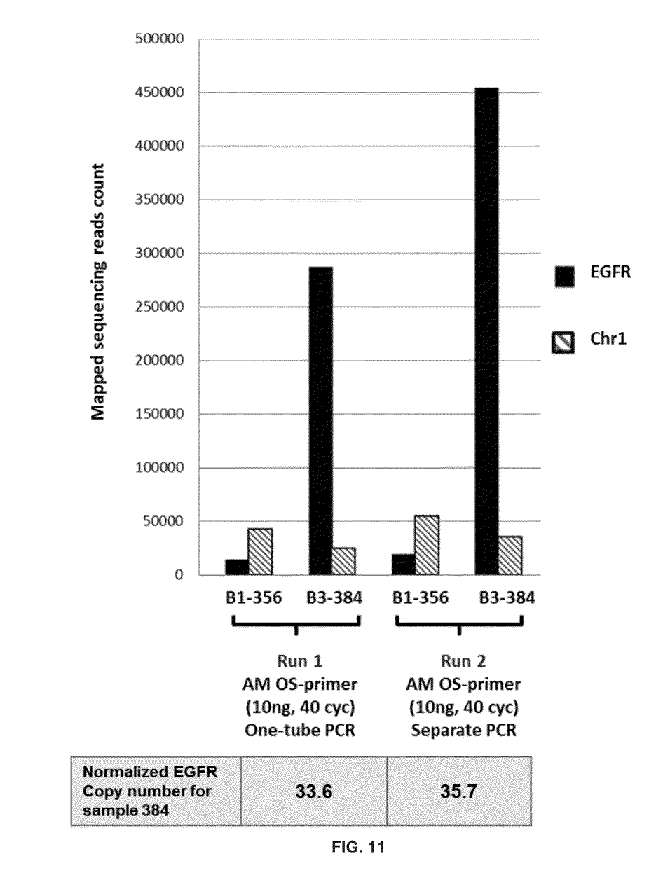

The barcode information was then used to associate the sequence read with the sample from which it was prepared (sample 384 or sample 356). The specific sequence reads from EGFR or from chromosome 1 were counted and normalized to assess relative copy number status of EGFR compared to the copy number of chromosome 1, which served as a control (FIG. 11).

In addition, sequence count data from sample 356 was used as a reference to determine the relative copy number of EGFR and chromosome 1. This relative copy number was then used to provide an adjustment factor for normalizing EGFR copy number for sample 384. The normalized EGFR copy numbers for sample 384 were 33.6 copies and 35.7 copies, respectively, for the two runs (FIG. 11).

### Example 6

### Hairpin Primers Comprising PEG

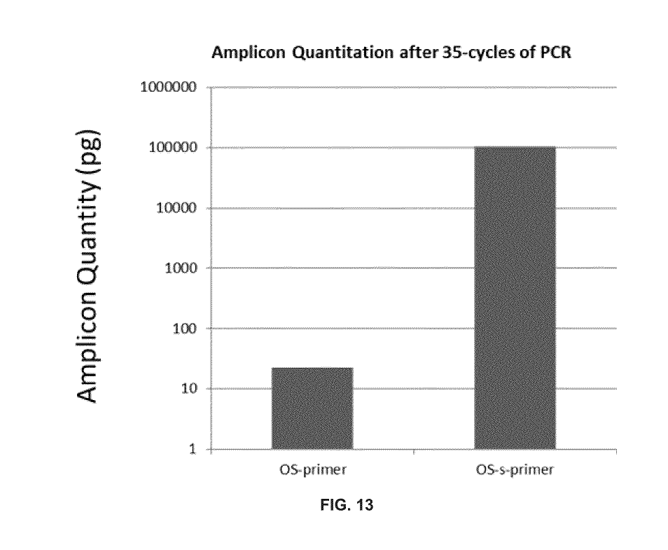

During the development of the technology provided herein, hairpin oligonucleotides comprising polyethylene glycol (PEG) linkers were designed (FIG. 12) and tested (FIG. 13). It was contemplated that hairpin oligonucleotides comprising PEG linkers would be useful for amplification reactions (e.g., as described herein) using a polymerase (e.g., a high-fidelity polymerase) that comprises a proof-reading activity, a 3′ exonuclease activity, and/or a strand displacement activity, but that lacks a 5′ exonuclease activity.

In these designs, the loop portion of the hairpin oligonucleotide primer comprises a PEG linker instead of linked nucleotides (FIG. 12). The DNA-PEG junction stops polymerase extension. In some embodiments, a hairpin oligonucleotide comprises a uracil residue, which provides for excision of portions of the hairpin oligonucleotide primers using an enzyme such as uracil-DNA glycosylase (UDG) and endonuclease VIII at appropriate stages of amplification to remove the PEG moiety in the final amplicon.

Experiments were conducted indicating that the PEG-based loop increases the hybridization and/or reaction kinetics during portions of the amplification reaction, which leads to increased efficiency of generating amplicons (FIG. 13). To compare amplification using hairpin primers with a PEG loop (FIG. 12, “OS-s-primer (PEG loop)”) and without a PEG loop (FIG. 12, “OS-primer (DNA loop)”), primers were designed and tested in amplification reactions. The two types of hairpin primers comprised the same single stranded priming region and the same duplex region except the PEG loop primer comprised a uracil residue “U” in the duplex. The PEG loop hairpin primer also comprised a uracil (“U”) near or adjacent to the loop region (see FIG. 12). Amplification with the PEG hairpin primer produced approximately 5000 to 10,000 more amplicons (as measured by mass in pg) than the equivalent hairpin primer that did not comprise the PEG loop (FIG. 13).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1-60. (canceled)

61. A hairpin oligonucleotide comprising:
a) a single-stranded region comprising an amplicon-specific priming segment;
b) a double-stranded duplex region comprising a first self-complementary region hybridized to a second self-complementary region;
c) a loop region;
d) a blocker moiety;
e) a fluorescent moiety; and
a quenching moiety,
wherein the second self-complementary region comprises the fluorescent moiety and the quenching moiety.

62. The hairpin oligonucleotide of claim 61 wherein the blocker moiety is at or near the junction of the single-stranded loop region and the double-stranded duplex region.

63. The hairpin oligonucleotide of claim 61 comprising a tag, an adaptor sequence, a universal sequence, and/or an index sequence.

64. The hairpin oligonucleotide of claim 63 wherein the tag comprises a linker, index, capture sequence, restriction site, primer binding site, and/or antigen.

65. The hairpin oligonucleotide of claim 61 wherein the loop region comprises a single-stranded loop region and/or a polyethylene glycol linker.

66. The hairpin oligonucleotide of claim 61 wherein the blocker moiety is exonuclease resistant.

67. The hairpin oligonucleotide of claim 61 wherein the blocker moiety is a phosphorothioate bond or a peptide-nucleic acid linkage.

68. The hairpin oligonucleotide of claim 61 wherein the fluorescent moiety is selected from the group consisting of xanthene, fluorescein, rhodamine, BODIPY, cyanine, coumarin, pyrene, phthalocyanine, FAM, VIC, JOE, Cy3, Cy5, Cy3.5, Cy5.5, TAMRA, ROX, HEX, and phycobiliprotein.

69. The hairpin oligonucleotide of claim 61 wherein the quenching moiety is a Black Hole Quencher or an Iowa Black Quencher.

70. The hairpin oligonucleotide of claim 61 wherein the double-stranded duplex region comprises a mismatch.

71. The hairpin oligonucleotide of claim 61 wherein the first self-complementary region and the second self-complementary region are not hybridized at or above a denaturing temperature in an amplification reaction and/or wherein the first self-complementary region and the second self-complementary region are hybridized below a denaturing temperature in an amplification reaction.

72. A method for producing a sequencing library comprising an amplicon, the method comprising:
a) providing a reaction mixture comprising a hairpin oligonucleotide according to claim 61 and a nucleic acid to be sequenced; and
b) exposing the reaction mixture to conditions appropriate for producing an amplicon.

73. The method according to claim 72 wherein the reaction mixture further comprises a polymerase comprising exonuclease activity.

74. The method according to claim 72 further comprising monitoring a fluorescence signal at the emission wavelength of the fluorescent moiety.

75. The method according to claim 72 further comprising providing a second primer, wherein the second primer is a hairpin oligonucleotide comprising:
a) a single-stranded region comprising an amplicon-specific priming segment;
b) a double-stranded duplex region comprising a first self-complementary region hybridized to a second self-complementary region;
c) a single-stranded loop region; and
d) a Mocker moiety.

76. The method according to claim 72 further comprising sequencing the amplicon to produce a nucleotide sequence, wherein the nucleotide sequence comprises sequence from the nucleic acid and an index sequence.

77. The method according to claim 72 further comprising associating the nucleotide sequence with a sample.

78. The method according to claim 72 further comprising mixing a first amplicon and a second amplicon to produce a multiplex sequencing library.

79. The method according to claim 72 further comprising quantifying an amount of amplicon to provide in a sequencing library.

80. A kit for generating a sequencing library comprising adaptor-tagged amplicons, the kit comprising:
a) a plurality of hairpin oligonucleotides according to claim 61, wherein each of said plurality of hairpin oligonucleotides comprises at least one of a plurality of index sequences; and
b) a polymerase comprising exonuclease activity.


SEQUENCE LISTING (22 sequences)

SEQ ID NO: 1 - DNA, 83 bp, Artificial Sequence
  Synthetic
tcaccgactgcccatagagaggaaagcgcctccgctttcctctctatgggcagtcggtgatccttcctttcatgctctcttcc

SEQ ID NO: 2 - DNA, 90 bp, Artificial Sequence
  Synthetic
ctgagtcggagacacgcagggatgaccatctcatccctgcgtgtctccgactcagctaaggtaacgatcttcctccatctcatagctgtc

SEQ ID NO: 3 - DNA, 84 bp, Artificial Sequence
  Synthetic
tcaccgactgcccatagagaggaaagcgcctccgctttcctctctatgggcagtcggtgatccaagtctgaatgaggtctgatg

SEQ ID NO: 4 - DNA, 90 bp, Artificial Sequence
  Synthetic
ctgagtcggagacacgcagggatgaccatctcatccctgcgtgtctccgactcagctaaggtaacgattgtgtctaatcaactggagacg

SEQ ID NO: 5 - DNA, 26 bp, Artificial Sequence
  Synthetic
ttatgtggtgacagatcacggctcgt

SEQ ID NO: 6 - DNA, 26 bp, Artificial Sequence
  Synthetic
accaaacttaggaacttgcctgccct

SEQ ID NO: 7 - DNA, 83 bp, Artificial Sequence
  Synthetic
tcaccgactgcccatagagaggaaagcgcctccgctttcctctctatgggcagtcggtgatcttcctccatctcatagctgtc

SEQ ID NO: 8 - DNA, 90 bp, Artificial Sequence
  Synthetic
ctgagtcggagacacgcagggatgaccatctcatccctgcgtgtctccgactcagctaaggtaacgatccttcctttcatgctctcttcc

SEQ ID NO: 9 - DNA, 83 bp, Artificial Sequence
  Synthetic
tcaccgactgcccatagagaggaaagcgcctccgctttcctctctatgggcagtcggtgattgtgtctaatcaactggagacg

SEQ ID NO: 10 - DNA, 91 bp, Artificial Sequence
  Synthetic
ctgagtcggagacacgcagggatgaccatctcatccctgcgtgtctccgactcagctaaggtaacgatccaagtctgaatgaggtctgatg

SEQ ID NO: 11 - DNA, 49 bp, Artificial Sequence
  Synthetic
cctctctatgggcagtcggtgatcatcaccttcctttcatgctctcttc

SEQ ID NO: 12 - DNA, 67 bp, Artificial Sequence
  Synthetic
ccatctcatccctgcgtgtctccgactcagctaaggtaacgattcttcctccatctcatagctgtcg

SEQ ID NO: 13 - DNA, 47 bp, Artificial Sequence
  Synthetic
cctctctatgggcagtcggtgattcttcctccatctcatagctgtcg

SEQ ID NO: 14 - DNA, 70 bp, Artificial Sequence
  Synthetic
ccatctcatccctgcgtgtctccgactcagctaaggtaacgatcatcaccttcctttcatgctctcttcc

SEQ ID NO: 15 - DNA, 49 bp, Artificial Sequence
  Synthetic
cctctctatgggcagtcggtgatcgccaagtctgaatgaggtctgatga

SEQ ID NO: 16 - DNA, 69 bp, Artificial Sequence
  Synthetic
ccatctcatccctgcgtgtctccgactcagctaaggtaacgataggctgtgtctaatcaactggagacg

SEQ ID NO: 17 - DNA, 49 bp, Artificial Sequence
  Synthetic
cctctctatgggcagtcggtgataggctgtgtctaatcaactggagacg

SEQ ID NO: 18 - DNA, 69 bp, Artificial Sequence
  Synthetic
ccatctcatccctgcgtgtctccgactcagctaaggtaacgatcgccaagtctgaatgaggtctgatga

SEQ ID NO: 19 - DNA, 90 bp, Artificial Sequence
  Synthetic
ctgagtcggagacacgcagggatgaccatctcatccctgcgtgtctccgactcagaagaggattcgatcttcctccatctcatagctgtc

SEQ ID NO: 20 - DNA, 90 bp, Artificial Sequence
  Synthetic
ctgagtcggagacacgcagggatgaccatctcatccctgcgtgtctccgactcagaagaggattcgatccttcctttcatgctctcttcc

SEQ ID NO: 21 - DNA, 90 bp, Artificial Sequence
  Synthetic
ctgagtcggagacacgcagggatgaccatctcatccctgcgtgtctccgactcagaagaggattcgattgtgtctaatcaactggagacg

SEQ ID NO: 22 - DNA, 91 bp, Artificial Sequence
  Synthetic
ctgagtcggagacacgcagggatgaccatctcatccctgcgtgtctccgactcagaagaggattcgatccaagtctgaatgaggtctgatg